(19) United States
(12) Patent Application Publication
(10) Pub. No.: US 2019/0367623 A1
(43) Pub. Date: Dec. 5, 2019

(54) METHODS OF TREATING NEURODEGENERATIVE DISEASES BY INDUCING DISEASE-ASSOCIATED MICROGLIA (DAM) CELLS

(71) Applicant: YEDA RESEARCH AND DEVELOPMENT CO. LTD., REHOVOT (IL)

(72) Inventors: Michal SCHWARTZ-EISENBACH, REHOVOT (IL); Ido AMIT, REHOVOT (IL); Hadas KEREN-SHAUL, REHOVOT (IL); Amit SPINRAD, REHOVOT (IL); Assaf WEINER, REHOVOT (IL); Orit MATCOVITCH NATHAN, REHOVOT (IL); Raz DVIR, REHOVOT (IL)

(21) Appl. No.: 16/478,179

(22) PCT Filed: Jan. 17, 2018
(86) PCT No.: PCT/IL2018/050062

§ 371 Date: Jul. 16, 2019

Related U.S. Application Data
(60) Provisional application No. 62/447,047, filed on Jan. 17, 2017.

(51) Int. Cl. C07K 16/28; C12N 15/113; A61P 25/28

(52) U.S. Cl. CPC C07K 16/2866; C07K 16/28; C12N 2310/531; A61P 25/28; C12N 2310/14; C12N 15/1138

(57) ABSTRACT

An active agent that causes an increase in the number of disease-associated microglia (DAM) for use in treating a neurodegenerative disease is provided.

### FIELD OF THE INVENTION

The present invention relates to methods for treatment of neurodegenerative disease.

### BACKGROUND

The central nervous system (CNS), as an immune privileged site, has evolved unique mechanisms to allow it to benefit from its resident myeloid cells, microglia, as well as from communication with the systemic immune system. In the mouse embryo, microglia migrate from the yolk sac to the CNS at embryonic days 8-9, and undergo a stepwise program of development that is synchronized with the brain developmental process and subsequently acquire a stable phenotype essential for the brain protection and homeostasis. Microglia immune activity is restrained by dedicated immune inhibitory pathways that suppress unwanted inflammatory responses and tissue destruction that are often associated with immune activation. These checkpoint mechanisms include direct inhibitory interactions of microglia with neurons, through the receptor-ligand pairs Cx3cl1-Cx3cr1 and CD200-CD200R, soluble molecules present in the CNS milieu (e.g. TGF-β), and intracellular regulators such as the transcription factor MafB. Nevertheless, these mechanisms may be counterproductive under extreme conditions when reparative microglial activity is needed.

Alzheimer's disease (AD) is an age-related neurodegenerative disease characterized by progressive memory decline and cognitive dysfunction, defined histologically by the parenchymal deposition of amyloid-beta (Aβ) plaques, the formation of neurofibrillary tangles and neuroinflammation. Numerous studies reported conflicting results regarding the contribution of systemic immunity, recruited monocytes and tissue resident microglia to AD onset and disease progression (Jay et al., 2015, Wang et al., 2016). Some reports show that under such conditions, microglia acquire pro-inflammatory activity, which has been associated with disease escalation (Wang et al., 2015). However, the current methods, analyzing bulk cell populations isolated based on a small set of surface markers, might be limited in resolving the heterogeneity, niche specificity and complexity of immune cell types within the CNS. Single-cell genomic technologies enable unbiased characterization of immune cell types and states, transitions from normal to disease and response to therapies, supporting comprehensive genome-wide sampling by single-cell RNA-seq as an effective tool to systematically resolve immune heterogeneity in AD. Single cell analysis can further identify potential markers, pathways and regulatory factors, promoting testable hypotheses to elucidate molecular mechanisms of immune regulation in AD.

Taken together, it is still not clear whether microglial function in neurodegenerative diseases is beneficial but insufficient, or whether these cells are effective at early disease stages but lose their efficacy. Importantly, the pathways and molecular mechanisms of microglia activity at the different stages of AD thereby remain controversial. Furthermore, there is still an unmet need for effective treatments of neurodegenerative diseases.

### SUMMARY OF THE INVENTION

In one aspect, the present invention provides an active agent that causes an increase in the number of disease-associated microglia (DAM) for use in treating a neurodegenerative disease.

In another aspect, the present invention provides an antibody against a microglial checkpoint molecule selected from Cx3cr1, Cx3CL1, Tmem119, P2ry12, CD200, CD200R, Ccr5, and P2ry13, for use in treating Alzheimer's disease, wherein said antibody is administered intracranially, intranasally or into the CSF via intrathecal injection, LP, CM, or ICV injection.

In a further aspect, the present invention provides an antibody against a microglial checkpoint molecule selected from Cx3cr1, Cx3CL1, Tmem119, P2ry12, CD200, CD200R, Ccr5, and P2ry13, for use in treating amyotrophic lateral sclerosis, wherein said antibody is administered intracranially, intranasally or into the CSF via intrathecal injection, LP, CM, or ICV injection.

### BRIEF DESCRIPTION OF THE DRAWINGS

The patent or application file contains at least one drawing executed in color. Copies of this patent or patent application publication with color drawings will be provided by the Patent Office upon request and payment of the necessary fee.

FIGS. 1A-1J show clustering of immune cells in the brain of an AD mouse model by a single cell RNA-seq analysis, revealing a unique microglia type associated with Alzheimer's disease. (A) Schematic diagram showing the isolation of single cells from brains of WT and Tg-AD (AD) mice for massively parallel single cell RNA-seq (MARS-seq) yielding genotype to cellular and molecular phenotype relations. (B) FACS gating strategy for isolating immune cells (CD45+) from brains of AD and WT mice. SSC-A, Side scatter (A: AD); FSC-A/W, Forward scatter (A: AD; W: WT) (C) Heatmap showing clustering analysis of 8016 single cells, featuring 220 most variable genes, from 3 WT and 3 AD 6 months old mice. The expression level (UMI count, see Methods) of selected marker genes for each cluster (I-X) is shown on the right. (D) Bar plots showing mean expression (UMI count) of selected genes across clusters I-X (clusters colored as in C) analyzed from the immune cells in the brains of WT and Tg-AD (AD) mice. (E) Dot plot showing the percentage of WT (beige) or AD (red) cells out of the total CD45+ cells in each of the clusters identified in C. Each data point, circle (female) or square (male), represents an independent single cell experiment performed on an individual animal. (F) t-SNE plot of the 8016 single cells depicting the separation into the ten clustered as shown in C. (G) Projection of selected genes onto the t-SNE plot depicting the ten immune clusters in WT and AD brains (corresponding to FIG. 1F). Number for each gene represents the max expression level (log2 UMI counts) depicted by a color bar (shown in FIG. 2G). (H) Volcano plot showing the fold change of genes (log2 scale) between DAM (microglia3) to homeostatis microglia (microglia1) from the AD male brain (x-axis) and their significance (y-axis, -loglog scale). Highly significant genes are indicated by a red dot. P-values were determined by Mann-Whitney U-test with FDR correction. (I) Volcano plot showing the fold change of genes (log2 scale) in two individual animals (females) between DAM (microglia3) to regular microglia (microglia1) from the AD brain (x-axis) and their significance (y-axis, -loglog scale). P-values were determined by Mann-Whitney U-test with FDR correction. (J) Gene Ontology (GO) analysis showing enrichment of GO terms in DAM associated genes.

FIGS. 2A-2M show that disease associated microglia (DAM) display dynamics of activation during AD progression. (A) FACS gating strategy using the CD11c positive cells for enriching the DAM population in AD brain. (B) Bar plot showing the relative distribution of each cluster identified in FIG. 1C with CD45+ sorting (left, 1045 cells) compared to CD11c+ microglia (CD45^{low}CD11b+) enrichment (right, 186 cells). (C) Heat map showing projection of CD11c+ microglia from 6-month old AD mice onto the corresponding CD45+ selection. (D) kNN projection of all CD11c+ cells across disease progression. In-silico filtering the monocytes (dark gray), leaving microglia (yellow) and DAM (red) for further analysis (E) kNN projection of the 893 single cells taken from the AD mouse at each time point along disease progression (1, 3, 6, 8 months; color) on the background of all microglia (grey). X-axis refers to the transition axis from homeostatic microglia to DAM. (F) Bar plots quantifying the percentage of cells presented in E along the activation axis. (G) Projection of key marker genes onto the graph plot of microglia. Color bar below each plot represents color scale level (log2 UMI counts). (H) Graph displaying sliding window smoothed expression (UMI counts, w=20) of selected markers (Cd9, Trem2) in each cell along the activation axis with a generalized logistic curve fit (marked in red). t_{1/2} represents half maximal response. (I) Heat map showing the kinetics of DAM differential genes ordered by their t_{1/2}. Top panel contains down regulated genes and lower panel contains up regulated genes. (J) Graphs showing the ordering of key marker genes along the transition axis. (K) Distribution of microglia, intermediate state (microglia2) and DAM in cells sorted at the four time points taken, for WT (CD11b+) and AD (CD11b+CD11c+) mice. (L) Scatter plot showing a 2Kb sliding window read coverage (log2) of H3K4me2 in microglia isolated from brains of AD model (x-axis) vs. DAM (y-axis). AD mice were 6.7-month old on average. See Methods for details. (M) Normalized profiles of H3K4me2 signals in 150 kilobase (kb) regions in the Lpl gene locus on chromosome 8, positions 71,361,500-71,508,500 analyzed in microglia from brains of 6 months WT mouse (WT microglia), microglia from AD mouse (AD microglia) and DAM from AD mouse (AD DAM).

FIGS. 3A-3K show that DAM cells are localized near AD plaques. (A) Projection of 6347 single cells isolated from either cortex (3247) or cerebellum (3100) collected from brains of 6 months old WT and Tg-AD (AD) mice (n=2 for each area) onto the t-SNE plot of FIG. 1F, showing the spatial location of DAM in the cortex of AD mice (indicated by an arrow). (B) Dot plot quantification for microglia clusters I-III (microglia1, microglia2 and microglia3—DAM) showing the percentage of WT (beige) or AD (red) cells out of the total CD45+ cells in cortex/cerebellum shown in A. Each data point (square, male) represents an independent single cell experiment performed on an individual animal. (C) Representative immunofluorescence images of CD11c (green) and microglia (IBA-1, red) in cortical sections, of 6-month old WT and Tg-AD (AD) mice. Cell nuclei are shown in blue (DAPI). Scale bar 10 μM. (D) Bar plot showing quantification of CD11c+ microglia out of total microglia (IBA1+), based on overall 944 cells counted in the cortex of WT and AD mice. ***<0.001. Error bars represent standard error of the mean. (E) Immunofluorescence imaging of CD11c positive microglia together with Aβ plaques (grey). Scale bar 10 μM. (F) Representative immunofluorescence images of CD11c (green), TIMP2 (cyan) and microglia (IBA-1, red) in cortical sections, of 6-month old WT and Tg-AD (AD) mice. Cell nuclei are shown in blue (DAPI). Scale bar 10 μM. Arrows indicate co-localization of TIMP2 and CD11c on IBA1 positive cells. (G) smFISH of mRNA molecules for Cx3cr1 (green), a marker for homeostatic microglia, and Csf1 (red), a DAM marker, together with Aβ plaques immunostaining (grey) and DAPI staining (blue) in intact brain tissue taken from 6-month old WT and AD mice. Imaging of AD mouse brains was performed in a region with low density of Aβ plaques (AD-Aβ distal; bottom left) and in a region with a high density of Aβ plaques (AD-Aβ proximal; right). Large yellow blobs are cytoplasmic auto-fluorescent objects. Scale bar 5 μM. (H) Left—quantification of the number of Csf1 molecules in microglia. Horizontal lines are medians, boxes demarcate the 25-75 percentiles; vertical lines are 1.5 times the interquartile range. Red plus signs represent outliers. Right—average UMI count obtained from the single cell RNA-seq data. Quantification was done based on the overall number and size of Aβ plaques in each field. n(WT)=39, n(AD-Aβ distal)=17, n(AD-Aβ proximal)=66, ***<0.001. ns=non-significant. Error bars represent standard error of the mean. (I) Dot plot quantification for microglia clusters I-X in the cortex/cerebellum (C/C) experiment, showing the percentage of WT (beige) or AD (red) cells out of the total CD45+ cells in each of the clusters shown in FIG. 3A. Each data point (square, male) represents an independent single cell experiment performed on an individual animal. (J) smFISH of mRNA molecules for Cx3cr1 (green), a marker for homeostatic microglia, and Lpl (red), a DAM marker, together with Aβ plaques immunostaining (grey) and DAPI staining (blue) in intact brain tissue taken from 6-month old WT and AD mice. Imaging of AD mouse brains was performed in a region with low density of Aβ plaques (AD-Aβ distal; bottom left) and in a region with a high density of Aβ plaques (AD-Aβ proximal; right). Large yellow blobs are cytoplasmic auto-fluorescent objects. Scale bar 5 μm. (K) Left—quantification of the number of Lpl molecules in microglia. Horizontal lines are medians, boxes demarcate the 25-75 percentiles; vertical lines are 1.5 times the interquartile range. Red plus signs represent outliers. Right—average UMI count obtained from the single cell RNA-seq data. Quantification was done based on the overall number and size of Aβ plaques in each field. n(WT)=34, n(AD-Aβ distal)=38, n(AD-Aβ proximal)=152, ***<0.001. ns=non-significant. Error bars represent standard error of the mean.

FIGS. 4A-4K show that DAM are phagocytic cells conserved in human and other neurodegenerative diseases. (A) Representative images from brain regions with low and high density of Aβ plaques (Aβ distal, top; Aβ proximal, bottom, respectively) of 6-month old Tg- AD mice, stained for a DAM marker gene (LPL; green), IBA-1 (microglia, red) and Thioflavin-S (ThioS; Aβ, gray). Small squares (inserts) represent position of images by cell nuclei staining (DAPI, blue). (B) Bar plot showing the fraction of IBA-1+ cells (microglia) in Aβ distal and proximal brain regions, stained for LPL and ThioS. (C) Representative image of postmortem AD human hippocampal section (n=5) stained for LPL (green), microglia (IBA-1, red) and Thioflavin-S (ThioS) for Aβ (gray). Four out of five samples stained positive for microglia expressing Lpl labeled with Aβ particles. All scale bars are 10 μm. (D) t-SNE plot of 3194 single cells taken from the spinal cord of WT and ALS mice at day 80 and day 135 of the disease. Microglia are shown in yellow, DAM are indicated in red and other immune cells are shown in shades of grey. (E) Representative image of postmortem AD human hippocampal section (n=5) and a human non-AD brain (n=3) stained for LPL (green), microglia (IBA-1, red) and Thioflavin-S (ThioS) for Aβ plaques (gray). Bar plots showing fraction of IBA-1+ cells (microglia) in Aβ distal and proximal regions in postmortem human AD brain (n=110 cells per region). Shown is a representative quantification of one postmortem brain out of three with a similar pattern. Scale bar 10 μm. (F) Heat map showing clustering analysis (k=13) of 3194 single cells, featuring 150 most variable genes, from WT and ALS mouse model. The color of each cluster corresponds to the relevant cluster analyzed from the brains of the AD model (immune cells analysis in FIG. 1C). (G) Projection of Hexb, Cx3cr1 and Lpl onto the t-distributed stochastic neighbor embedding (t-SNE) plot depicting the immune cells in an ALS model. Number for each gene represents the max expression level (log2 UMI counts) depicted by a color bar (similar to FIG. 2G). (H) Scatter plot comparing z-statistics (Mann-Whitney U-test) for the differentially expressed genes in DAM vs. resting microglia in ALS (x-axis) vs. AD (y-axis). (I) Bar plots comparing the percentage of DAM in WT and ALS mice at day 80 and day 135 of the disease. (J) Scatter plot showing the average molecules count (log2 scale) of DAM in 5XFAD (x-axis) compared with DAM in the ALS model (y-axis). (K) Bar plots showing the number of microglia cells present in young (7 weeks) and old (20 months) mice along the activation axis. Grey region marks DAM activation area.

FIGS. 5A-5G show that DAM activation is initiated by a Trem2 independent pathway. (A) Projection of microglia on a kNN graph plot displaying the transition from homeostatic microglia (yellow) to stage 1 DAM (orange) and stage 2 DAM (red). The microglia cells (3864 cells) were extracted from whole brains of 6 months old WT, 5XFAD, Trem2^{-/-} and Trem2^{-/-} 5XFAD mice. (n=4). (B) Projection of the mice genotype on the model from A. Cells enriched for CD11b (microglia; CD45^{low}CD11b+) are marked in blue, cells enriched for CD11c (DAM; CD45^{low}CD11b+CD11c+) are marked in green. (C) The expression level (UMI counts) of selected marker genes in the different trem2 genotypes at the three microglia states: resting (homeostatic), stage 1 DAM and stage 2 DAM. (D) Bar plots showing mean expression (UMI count) of selected genes across clusters I-III: Homeostatic microglia (yellow), stage 1 DAM (orange) and stage 2 DAM (red). (E) Scatter plot showing the average molecules count (log2 scale) of resting (homeostatic) microglia in the Trem2^{-/-} (x-axis) compared with the WT (y-axis). (F) Scatter plot showing the average molecules count (log2 scale) of stage 1 DAM in the Trem2^{-/-} 5XFAD (x-axis) compared with the 5XFAD (y-axis). (G) Comparison of log p-values of the differential expression of stage 1 DAM vs. resting (homeostatic) microglia in the Trem2^{-/-} 5XFAD (x-axis) to the differential expression of stage 2 DAM vs. resting (homeostatic) microglia in 5XFAD (y-axis), log p-values sign corresponds to up-regulation or down-regulation.

FIG. 6 show that DAM are regulated through a two-step activation mechanism. Schematic illustration showing microglia switching from homeostatic to stage 1 DAM (Trem2 independent) and stage 2 DAM (Trem2 dependent) following signals such as those associated with AD pathology, aging and ALS pathology. Key genes involved in each stage are shown below each condition. Arrows indicate up (red) or down (green) regulation of the gene in the specific stage.

### DETAILED DESCRIPTION

Immune cells and pathways are frequently implicated in neurodegenerative conditions. For the last decades, the nature of the involvement of microglia, as well as infiltrating immune cells, in the response to brain pathologies have been under constant debate. This was partly due to technical limitations of using marker based approaches analyzing heterogeneous cell populations, which made it difficult to accurately define the immune cell types and states involved in brain homeostasis and disease. As a result, various innate immune functions have been interchangeably attributed to either microglia or infiltrating blood-derived monocytes. For example, in Alzheimer's disease (AD), as in many other neurodegenerative diseases, the local neuroinflammation that is associated with cytotoxicity and disease escalation has often been attributed mainly to microglia (Wang et al., 2016, Wang et al., 2015, Yuan et al., 2016). In contrast, several studies have attributed a positive role to infiltrating monocytes in the clearance of toxins from the brain (Jay et al., 2015, Baruch et al., 2015, Baruch et al., 2016).

In the present invention, the inventors were able to overcome many of these limitations, and avoid marker-based classifications, by using single cell RNA sequencing for sampling single immune cells from the brain. This work establishes a new experimental and conceptual paradigm to accurately analyze the involvement of the immune system in neurodegeneration and other brain pathologies. A novel subtype of protective microglia, disease-associated microglia (DAM), is identified in this work, and its dynamics along the course of the disease progression is revealed. Using immunohistochemistry and smFISH, the inventors have found that DAM are spatially associated with sites of AD pathology. Further analysis identifies DAM in another mouse model of neurodegenerative disease, namely, amyotrophic lateral sclerosis (ALS). Closer examination of the genes expressed by DAM along disease progression revealed elevation of lipid metabolism pathways, and phagocytic-related genes, corresponding to the need for plaque clearance in AD. Importantly, the DAM-associated program that was identified here includes genes that encode a large number of known risk factors which contribute to disease mitigation. While they were previously identified in AD, they were not functionally linked with microglia. Among these genes is Lpl, whose mutations are associated with an aggressive form of AD (Baum et al., 1999). The present study further shows that the expression of Lpl and additional risk factor genes occurs in the Trem2 dependent phase and attributes a new role for microglia function of these genes in AD. Immunostaining of DAM specific genes together with Aβ plaques identified enhanced phagocytic activity of DAM cells and their functional conservation in both mice and in human AD brains.

Trem2 is a microglia specific immunoreceptor, whose genetic variants are associated with increased risk of neurodegenerative diseases, and homozygous Trem2 loss of function mutations cause dementia in humans (Colonna and Wang, 2016). In addition, a rare variant of Trem2 has been associated with an increased risk for AD (Guerreiro et al., 2013, Jonsson et al., 2013, Song et al., 2016). In addition to Trem2, the inventors have now identified strong induction of Tyrobp in DAM. Tyrobp and Trem2 form a signaling complex, associated with Aβ clearance, that could enhance phagocytic activity of microglia, are associated with Aβ clearance, and participate in suppression of inflammatory responses by repression of microglia-mediated cytokine production and secretion (Ma et al., 2015). Here, using TREM2^{-/-} 5XFAD mice, the inventors were able to identify two sequential but distinct stages in DAM activation. The first step, which is Trem2 independent, involves activation of a set of genes, including the Trem2 signaling adaptor Tyrobp, Apoe and B2m, concomitantly with downregulation of microglia homeostatic molecules, e.g., Cx3cr1 and P2ry12 or P2ry13. The second phase of DAM activation, including induction of lipid metabolism and phagocytic pathways (e.g. Lpl, Cst7 and CD9) was found to be Trem2 dependent: this transition to fully activated DAM does not occur in the absence of the Trem2 receptor.

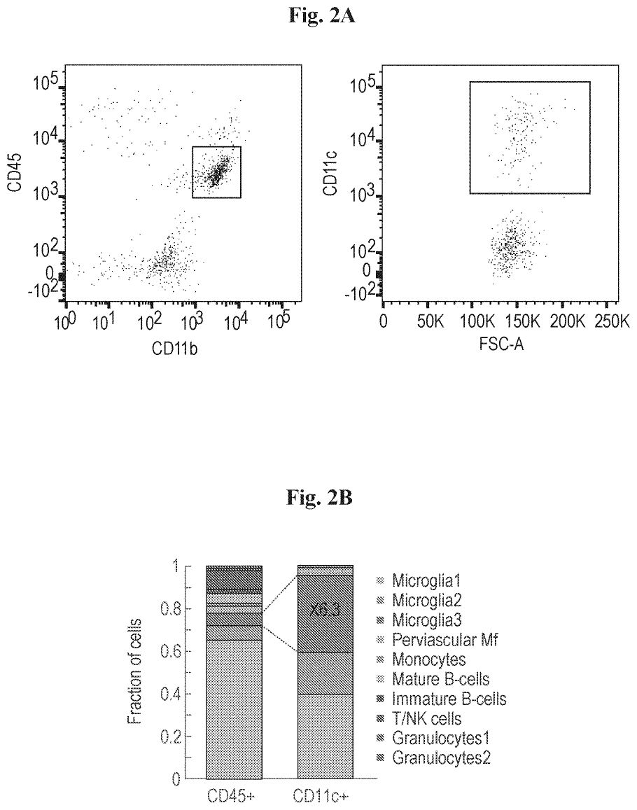

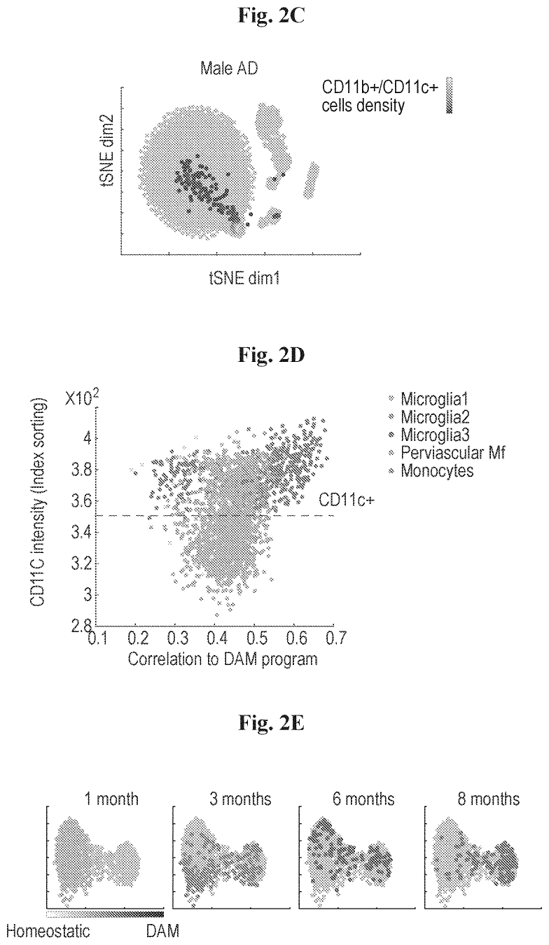

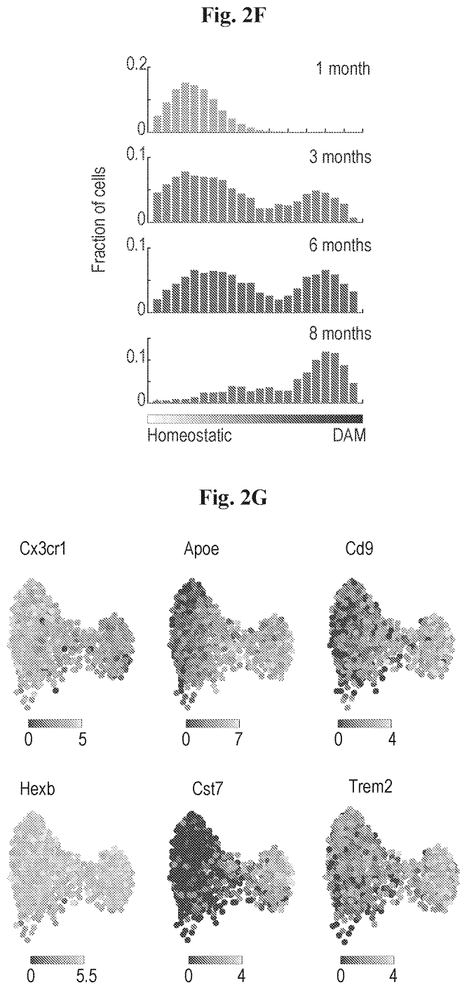

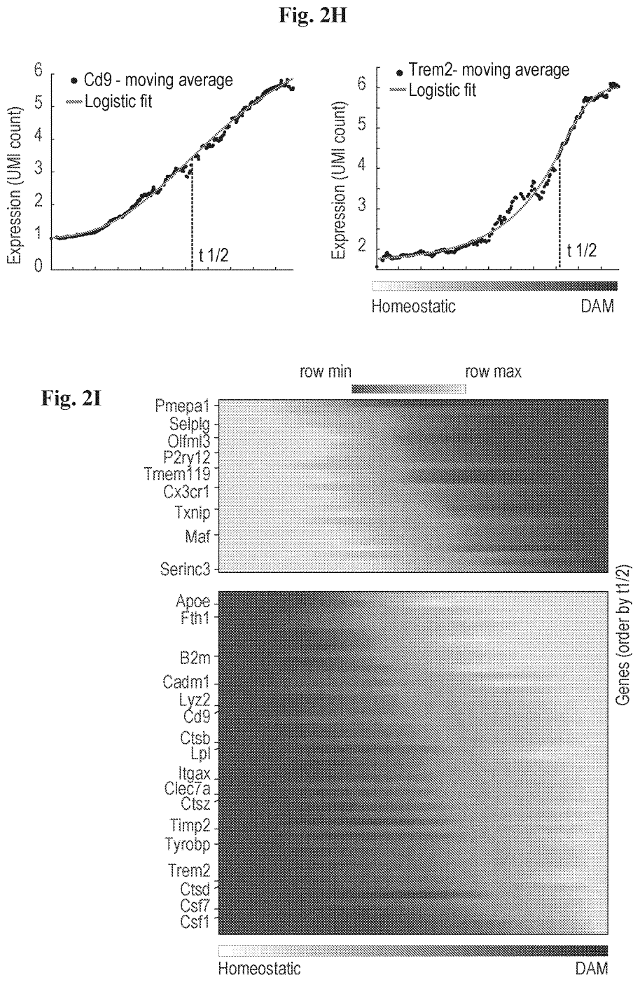

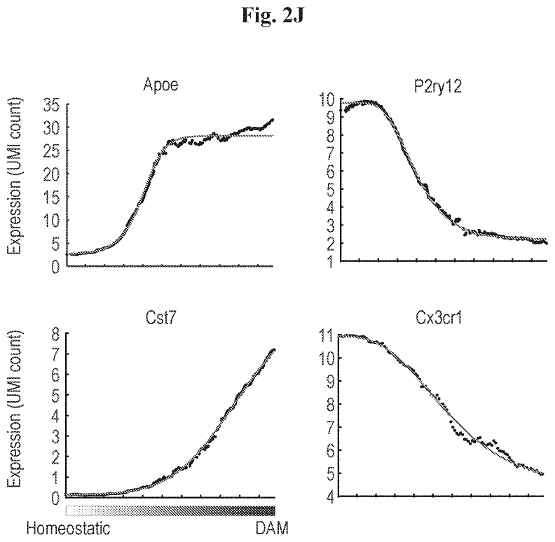

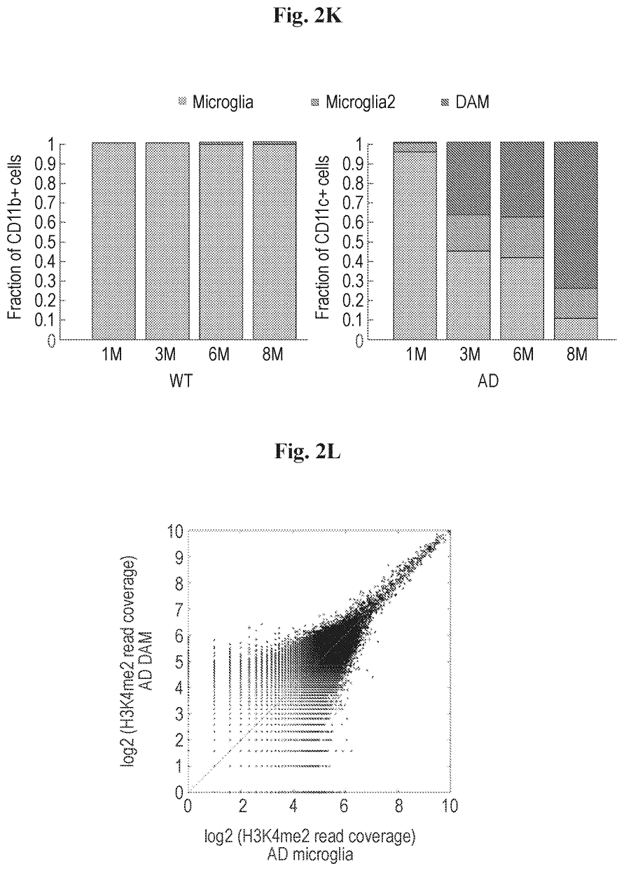

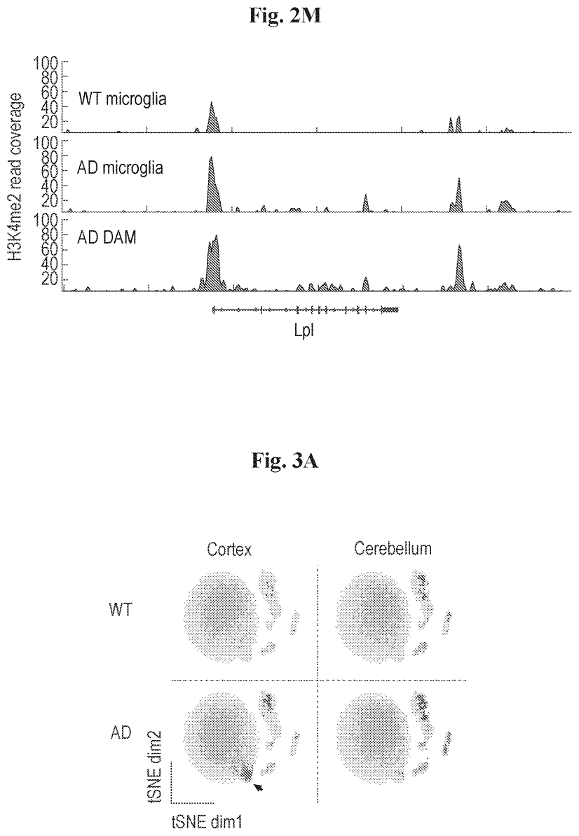

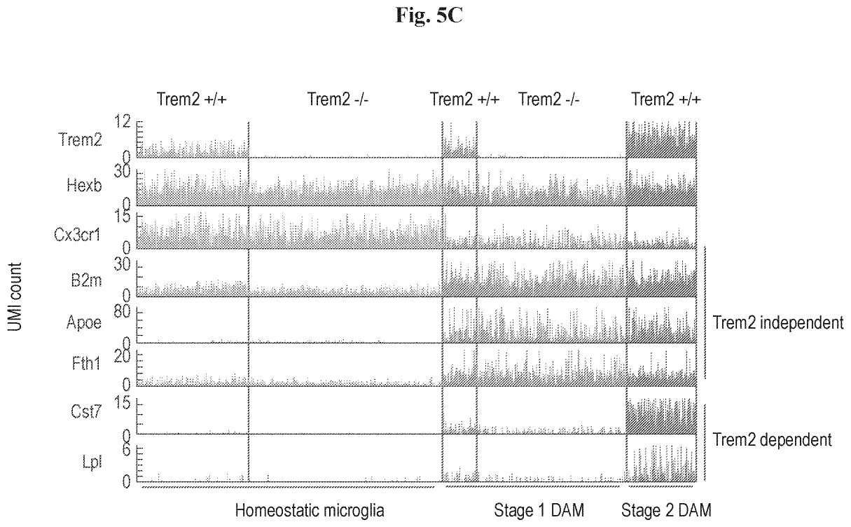

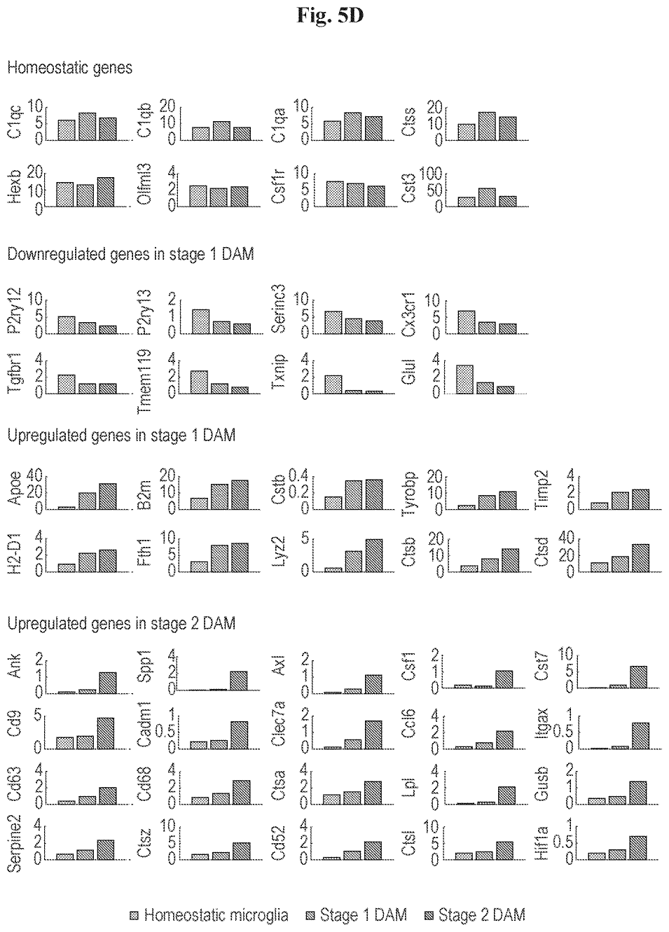

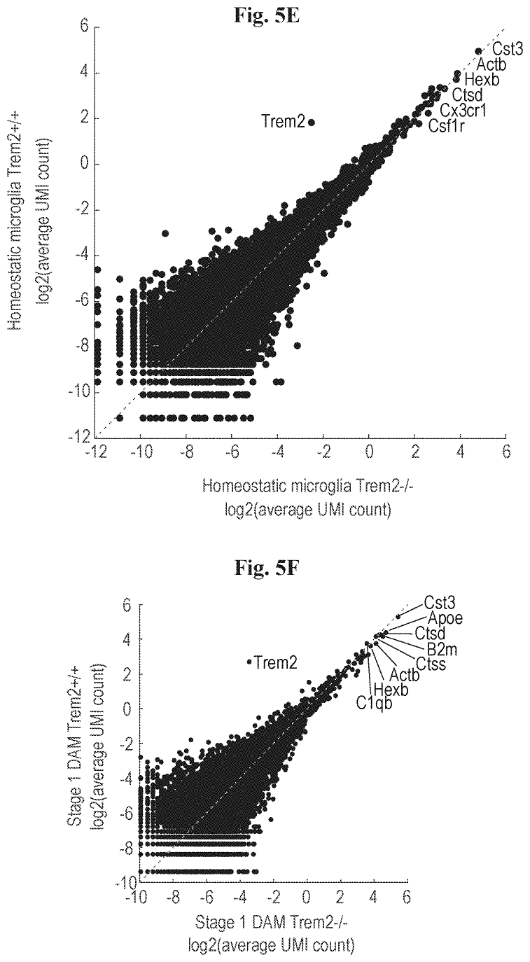

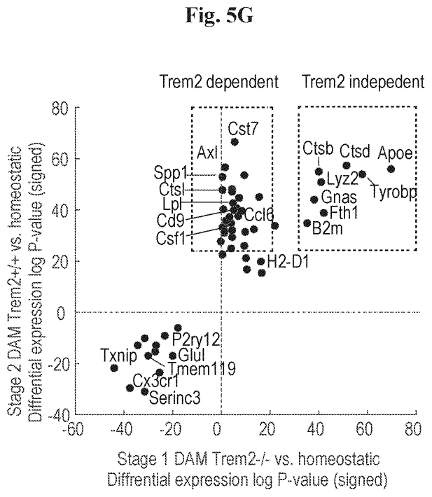

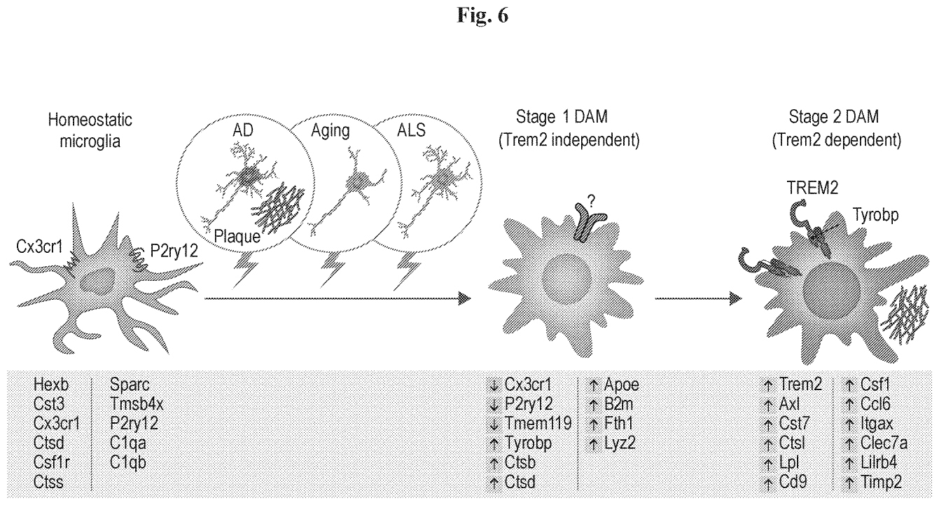

Taken together, these results shed new light on the role of microglia in combating AD, and suggest that at least a sub-population of microglia, the DAM, are beneficial for AD. It is possible that there is a trade-off in brain homeostasis between the number of DAM with phagocytic activity, and checkpoint mechanisms that keep them under tight control, e.g Cx3cr1 inhibitory signaling. Such checkpoint mechanisms, while essential for the function of microglia, for ensuring their risk-free immune activation, may become a negative factor when this balance is tilted and a strong phagocytic activity is needed in deficiency in Cx3CR1 signaling or under detrimental genetic backgrounds. In line with this hypothesis, it is shown here (e.g. FIG. 2I, FIG. 5D) that the elevation of the expression of genes associated with the first step of DAM activation is tightly coupled with the loss of expression of microglial homeostatic genes. Our results also support the observation that deficiency in Cx3CR1 signaling in AD animal models, led to a reduction in Aβ deposition (Lee et al., 2010).

The present invention is therefore directed to targeting the microglia specific inhibitory checkpoints to induce early DAM activation, and may be adopted as a therapeutic (or preventive) target to trigger a microglial response against AD plaque pathology, ageing and other neurodegenerative diseases.

Generalizing our study to other neurodegenerative diseases, we found a similar DAM subpopulation in an amyotrophic lateral sclerosis (ALS) mouse model. This suggests that DAM are not associated with the specific primary cause of disease pathology or disease etiology, but rather with a general program that is involved in clearance of the protease-resistant misfolded and aggregated proteins that commonly accumulate in neurodegenerative diseases and general aging-induced damage. In view of this the present invention is directed to providing a therapy that unleashes the ability of resident microglia to combat neurodegenerative disorders by increasing the number of DAM. The present application achieves this goal by blocking or otherwise circumventing microglia-specific checkpoint molecules.

Accordingly, in one aspect, the present invention provides a method for treating a neurodegenerative disease comprising administering to an individual in need thereof at least one active agent that causes an increase in the number of disease-associated microglia (DAM).

In another aspect, the present invention provides at least one active agent that causes an increase in the number of disease-associated microglia (DAM) for use in treating a neurodegenerative disease.

In an additional aspect, the present invention is directed to use of at least one active agent that causes an increase in the number of disease-associated microglia (DAM) for the preparation of a medicament for treatment of a neurodegenerative disease.

The term “disease-associated microglia” as used herein refers to microglia that exhibit increased expression of ApoE3, Cst7, Lpl, Tyrobp, CD9, B2m and Trem2, and decreased expression of microglia homeostatic molecules Cx3cr1 and P2ry12 or P2ry13 as compared with resting microglia.

In some embodiments, the at least one active agent causes an increase in the number of DAM by releasing or circumventing a restraint imposed on microglia immune activity by at least one microglia checkpoint molecule.

In some embodiments, the increase in the number of DAM is associated with an increase in microglia phagocytic activity without inflammatory activity. This can easily be assessed by methods well known in the art e.g. FACS analysis for phagocytic activity.

As shown in the present application, the expression of several molecules is reduced in the DAM phenotype as opposed to the homeostatic microglia (e.g. see FIGS. 2G, 2I, 2J, 5D). Such molecules are defined herein as “microglia homeostatic molecules” or “microglia checkpoint molecules”, since they prevent microglia from proceeding to become the immunologically active DAM cells. The microglia checkpoint molecules found include Cx3cr1, P2ry12, P2ry13, Ccr5, Tmem119, Calm2, Cd164, Cmtm6, Crybb1, Csf1r, Ecscr, Fscn1, Glul, Gpr56, Ifngr1, Lpcat2, Lrba, Lyn, Maf, Marcks, Olfml3, Pmepa1, Ptgs1, Rhob, Selplg, Serinc3, Slco2b1, Sparc, Srgap2, Txnip, and Zfhx3.

Accordingly, in some embodiments, the at least one microglia checkpoint molecule is a microglia homeostatic molecule selected from the group consisting of Cx3cr1, CD200, CD200R, P2ry12, P2ry13, Ccr5, Tmem119, Calm2, Cd164, Cmtm6, Crybb , Ecscr, Fscn1, Glul, Gpr56, Ifngr1, Lpcat2, Lrba, Lyn, Maf, Marcks, Olfml3, Pmepa1, Ptgs1, Rhob, Slco2b1, Selplg, Serinc3, Sparc, Srgap2, Txnip, and Zfhx3.

In some embodiments, the at least one microglia checkpoint molecule is selected from the group consisting of Cx3cr1, Tmem119, P2ry12, CD200, CD200R, Ccr5, Cd164, Zfhx3, Srgap2, Txnip, Ifngr1, P2ry13, Fscn1, Rhob, Cmtm6 and Gpr56.

Blocking these microglia checkpoint or homeostatic molecules, at the expression or at the activity level, is expected to enhance the DAM phenotype thereby increasing the number of DAM. Blocking at the activity level can be done by a binding molecule such as an antagonist to microglia homeostatic molecules, for example by an antibody to, or small molecule inhibiting, this molecule. The antibody or other antagonist binding molecule blocks or inactivates, or otherwise neutralizes the activity of the homeostatic molecule to stop or attenuate its effect, thereby causing the cells to progress to the DAM phenotype.

It is noted that some of the above-listed microglia checkpoint molecules have partners such that their effect is achieved by complexing with the partners. Accordingly, blocking of activity or inhibiting the expression of the partner protein will have a similar effect as blocking activity or inhibiting expression of the microglia checkpoint protein. Examples for such proteins are CD200R (partner of CD200), and Cx3cL1 (partner of Cx3cr1). Accordingly, blocking the activity or reducing the expression of partners of the above-listed microglia checkpoint molecules is also within the scope of the present invention.

In some embodiments, the at least one active agent releases a restraint imposed on said microglia by blocking, inhibiting or attenuating the activity of the at least one microglia checkpoint molecule.

Accordingly, in some embodiments, the at least one active agent is a binding-molecule capable of selectively binding and blocking or neutralizing at least one specific microglia checkpoint gene, said binding molecule is selected from the group consisting of an antibody, affibody, single-domain antibody (nanobody), single chain variable fragment (scFv), affilin, affimer, affitin, alphabody, anticalin, avimer, DARPin, Kunitz domain peptide and monobody.

More specifically, in some embodiments the binding-molecule is at least one antibody selected from the group consisting of anti-Cx3cr1; anti-Cx3CL1; anti-Tmem119; anti-P2ry12; anti-CD200; anti-CD200R; anti-Ccr5; anti-Cd164; anti-Zfhx3; anti-Srgap2; anti-Txnip; anti-Ifngr1; anti-P2ry13 ; anti-Fscn1; anti-Rhob; anti-Cmtm6; and anti-Gpr56 antibody.

Methods for identifying and/or characterizing microglia checkpoint inhibitors, i.e. an assay for determining if an active agent releases a restraint imposed on said microglia by blockade of said at least one microglia checkpoint are based on generic methods well known in the art of molecular biology and high-throughput screen. For example, microglia expressing the microglia checkpoint of interest are contacted in vitro with the tested antibodies or small molecules and binding to the microglia checkpoint of interest is assessed. Antibodies and small molecules that bind to the microglia checkpoint of interest are further characterized for inhibiting activity for example by measuring parameters showing activation of the microglia, such as upregulation of phagocytic and lipid metabolism genes such as Cst7 and Lpl.

Alternatively, in some embodiments the active agent releases a restraint imposed on said microglia by reducing the expression of at least one of the at least one microglia checkpoint molecule.

Methods for determining if an active agent reduces expression of microglia checkpoint molecules are based on generic methods well known in the art of molecular biology. For example, microglia expressing the microglia checkpoint of interest are contacted in vitro with a nucleic acid molecule that reduces the gene expression level of at least one gene encoding a microglia homeostatic molecule and are characterized for inhibiting activity for example by measuring parameters showing activation of the microglia, such as upregulation of phagocytic and lipid metabolism genes such as Cst7 and Lpl. The nucleic acid molecule may be an shRNA or siRNA comprising a nucleic acid sequence being complementary to a sequence within a nucleic acid sequence encoding said microglia checkpoint molecule, and the shRNA may be comprised within a vector, such as a modified lentivirus.

For example, in some embodiments, the at least one active agent is at least one nucleic acid molecule that reduces the gene expression level of at least one gene encoding a microglia homeostatic molecule selected from the group consisting of Cx3cr1, Cx3CL1, Tmem119; P2ry12; CD200; CD200R; Ccr5; Cd164; Zfhx3; Srgap2; Txnip; Ifngr1; P2ry13; Fscn1; Rhob; Cmtm6; and Gpr56.

In particular, the microglia homeostatic molecule in any one of the above mentioned embodiments is a human microglia homeostatic molecule.

In some embodiments, the at least one nucleic acid molecule is an isolated shRNA or artificial siRNA molecule comprising a nucleic acid sequence being complementary to a sequence within a nucleic acid sequence encoding said microglia checkpoint molecule, or a nucleic acid molecule encoding said isolated shRNA or artificial siRNA molecule. In some embodiments, the isolated/artificial siRNA or shRNA molecule comprises a nucleic acid sequence having a sequence identity of 90% or more, e.g. 95% or more, 98% or more, or 99% identity to one of the nucleic acid sequences encoding said microglia checkpoint molecules.

In some embodiments, the siRNA or shRNA molecule comprises a nucleic acid sequence being perfectly complementary to a sequence within the nucleic acid sequence encoding said at least one microglia checkpoint molecule.

In some embodiments, the nucleic acid molecule is comprised within a vector.

In some embodiments, the vector is a modified virus derived from a virus selected from the group consisting of a retrovirus, adenovirus, adeno-associated virus, pox virus, alphavirus, herpes virus and lentivirus.

In some embodiments, the vector is a modified lentivirus.

As shown in the present application, the expression of several molecules is increased in the DAM phenotype as opposed to the homeostatic microglia (e.g. see FIGS. 2G-2J, 5D). Such molecules are defined herein as “DAM-associated molecules” and include ApoE3, Cst7, Lpl, Tyrobp, CD9, and Trem2. It is possible that the activity of at least some of these molecules is downstream of the microglia checkpoints listed above, and their enhanced level of expression directly results from the lower expression level of the microglia checkpoints, or in other words, from release of the restraint or block imposed on the microglia by the checkpoint. Another possibility is that they are upstream from the microglia checkpoints and their activity causes downregulation of the microglia checkpoints. Either way, increasing the activity or the expression of DAM-associated molecules is also expected to result in or contribute to the DAM phenotype.

Accordingly, in some embodiments, the active agent increases the activity of, or up-regulates, at least one DAM-associated molecule.

Methods for identifying and/or characterizing an active agent that releases a restraint imposed on said microglia by increasing the activity of a DAM-associated molecule are based on generic methods well known in the art of molecular biology and high-throughput screen. For example, microglia expressing the microglia checkpoint of interest are contacted in vitro with antibodies or small molecules and binding to the DAM-associated molecule of interest is assessed. Antibodies and small molecules that bind to the DAM-associated molecule of interest are further characterized for agonist activity for example by measuring parameters showing activation of the microglia, such as upregulation of phagocytic and lipid metabolism genes such as Cst7 and Lpl.

In some embodiments the at least one active agent is at least one agonist increasing the activity of, or at least one nucleic acid molecule upregulating at least one gene encoding, at least one DAM-associated molecule selected from the group consisting of Trem2, ApoE3, Cst7, Lpl, Tyrobp, and CD9, but excluding ApoE4.

In some embodiments, the neurodegenerative disease is selected from the group consisting of Alzheimer's disease, amyotrophic lateral sclerosis, Parkinson's disease, Huntington's disease and age-related macular degeneration.

In some embodiments, the disease is Alzheimer's disease.

The method, active agent and pharmaceutical composition of the present invention may be for use in improving CNS motor and/or cognitive function, for example for use in alleviating age-associated loss of cognitive function, which may occur in individuals free of a diagnosed disease, as well as in people suffering from neurodegenerative disease.

Furthermore, the method, active agent and pharmaceutical composition may be for use in alleviating loss of cognitive function resulting from acute stress or traumatic episode. The cognitive function mentioned herein above may comprise learning, memory or both.

The term “CNS function” as used herein refers, inter alia, to receiving and processing sensory information, thinking, learning, memorizing, perceiving, producing and understanding language, controlling motor function and auditory and visual responses, maintaining balance and equilibrium, movement coordination, the conduction of sensory information and controlling such autonomic functions as breathing, heart rate, and digestion. The terms “cognition”, “cognitive function” and “cognitive performance” are used herein interchangeably and are related to any mental process or state that involves but is not limited to learning, memory, creation of imagery, thinking, awareness, reasoning, spatial ability, speech and language skills, language acquisition and capacity for judgment attention. Cognition is formed in multiple areas of the brain such as hippocampus, cortex and other brain structures. However, it is assumed that long term memories are stored at least in part in the cortex and it is known that sensory information is acquired, consolidated and retrieved by a specific cortical structure, the gustatory cortex, which resides within the insular cortex. In humans, cognitive function may be measured by any know method, for example and without limitation, by the clinical global impression of change scale (CIBIC-plus scale); the Mini Mental State Exam (MMSE); the Neuropsychiatric Inventory (NPI); the Clinical Dementia Rating Scale (CDR); the Cambridge Neuropsychological Test Automated Battery (CANTAB) or the Sandoz Clinical Assessment-Geriatric (SCAG). Cognitive function may also be measured indirectly using imaging techniques such as Positron Emission Tomography (PET), functional magnetic resonance imaging (fMRI), Single Photon Emission Computed Tomography (SPECT), or any other imaging technique that allows one to measure brain function.

An improvement of one or more of the processes affecting the cognition in a patient will signify an improvement of the cognitive function in said patient, thus in certain embodiments improving cognition comprises improving learning, plasticity, and/or long term memory. The terms “improving” and “enhancing” may be used interchangeably.

The term “learning” relates to acquiring or gaining new, or modifying and reinforcing, existing knowledge, behaviors, skills, values, or preferences.

The term “plasticity” relates to synaptic plasticity, brain plasticity or neuroplasticity associated with the ability of the brain to change with learning, and to change the already acquired memory. One measurable parameter reflecting plasticity is memory extinction.

The term “memory” relates to the process in which information is encoded, stored, and retrieved. Memory has three distinguishable categories: sensory memory, short-term memory, and long-term memory.

The term “long term memory” is the ability to keep information for a long or unlimited period of time. Long term memory comprises two major divisions: explicit memory (declarative memory) and implicit memory (non-declarative memory). Long term memory is achieved by memory consolidation which is a category of processes that stabilize a memory trace after its initial acquisition. Consolidation is distinguished into two specific processes, synaptic consolidation, which occurs within the first few hours after learning, and system consolidation, where hippocampus-dependent memories become independent of the hippocampus over a period of weeks to years.

In some embodiments, the at least one active agent is administered intracranially; intranasally; or into the cerebrospinal fluid (CSF) via intrathecal injection, lumbar puncture (LP), injection through the Cisterna Magna (CM), intracerebroventricular (ICV) injection.

In another aspect, the present invention provides a method for treating Alzheimer's disease comprising administering to an individual in need thereof an antibody against a microglial homeostatic molecule selected from Cx3cr1, CD200, Tmem119, Ccr5, P2ry12, and P2ry13, wherein said antibody is administered intracranially, intranasally or into the CSF via intrathecal injection, lumbar puncture (LP), injection through the Cisterna Magna (CM), intracerebroventricular (ICV) injection.

In another further aspect, the present invention provides a method for treating amyotrophic lateral sclerosis disease comprising administering to an individual in need thereof an antibody against a microglial homeostatic molecule selected from Cx3cr1, CD200, Tmem119, Ccr5, P2ry12, and P2ry13, wherein said antibody is administered intracranially, intranasally or into the CSF via intrathecal injection, lumbar puncture (LP), injection through the Cisterna Magna (CM), intracerebroventricular (ICV) injection.

In still an additional aspect, the present invention provides an antibody against a microglial homeostatic molecule selected from Cx3cr1, CD200, Tmem119, Ccr5, P2ry12, and P2ry13, for use in treating Alzheimer's disease, wherein said antibody is administered intracranially, intranasally or into the CSF via intrathecal injection, lumbar puncture (LP), injection through the Cisterna Magna (CM), intracerebroventricular (ICV) injection.

In yet an additional aspect, the present invention provides an antibody against a microglial homeostatic molecule selected from Cx3cr1, CD200, Tmem119, Ccr5, P2ry12, and P2ry13, for use in treating amyotrophic lateral sclerosis disease, wherein said antibody is administered intracranially, intranasally or into the CSF via intrathecal injection, lumbar puncture (LP), injection through the Cisterna Magna (CM), intracerebroventricular (ICV) injection.

In a related aspect, the present invention provides one or more nucleic acid molecule that reduces the gene expression level of at least one gene encoding a microglial homeostatic molecule selected from Cx3cr1, CD200, Tmem119, Ccr5, P2ry12, and P2ry13, for use in treating Alzheimer's disease, wherein said one or more nucleic acid molecule is administered intracranially, intranasally or into the CSF via intrathecal injection, lumbar puncture (LP), injection through the Cisterna Magna (CM), intracerebroventricular (ICV) injection. In another related aspect, the present invention provides one or more nucleic acid molecule that reduces the gene expression level of at least one gene encoding a microglial homeostatic molecule selected amyotrophic lateral sclerosis, wherein said one or more nucleic acid molecule is administered intracranially, intranasally or into the CSF via intrathecal injection, lumbar puncture (LP), injection through the Cisterna Magna (CM), intracerebroventricular (ICV) injection.

In an additional related aspect, the present invention provides at least one agonist increasing activity of, or at least one nucleic acid molecule upregulating at least one gene encoding, at least one DAM-associated molecule selected from the group consisting of Trem2, ApoE3, Cst7, Lpl, Tyrobp, and CD9, but excluding ApoE4, for use in treating Alzheimer's disease, wherein said at least one agonist or at least one nucleic acid molecule is administered intracranially, intranasally or into the CSF via intrathecal injection, lumbar puncture (LP), injection through the Cisterna Magna (CM), intracerebroventricular (ICV) injection.

In yet an additional related aspect, the present invention provides at least one agonist increasing activity of, or at least one nucleic acid molecule upregulating at least one gene encoding, at least one DAM-associated molecule selected from the group consisting of Trem2, ApoE3, Cst7, Lpl, Tyrobp, and CD9, but excluding ApoE4, for use in treating amyotrophic lateral sclerosis, wherein said at least one agonist or at least one nucleic acid molecule is administered intracranially, intranasally or into the CSF via intrathecal injection, lumbar puncture (LP), injection through the Cisterna Magna (CM), intracerebroventricular (ICV) injection.

In all aspects and embodiments of the application, the active agent may be a single active agent.

However, it is also conceivable that the active agent comprises a combination of active agents as defined in the application, for example: a combination of two or more agents that block the activity of microglia checkpoint molecules; a combination of two or more agents that reduce the expression of microglia checkpoint molecules; a combination of two or more agents that increase the activity of DAM-associated molecules; or a combination of two or more agents that upregulate the expression DAM-associated molecules.

Additionally combinations of active agents are also included within the invention: a combination of at least one agent that blocks the activity of microglia checkpoint molecules together with at least one agent that reduces the expression of microglia checkpoint molecules; a combination of at least one agent that increases activity of DAM-associated molecules together with at least one agent that upregulates the expression of DAM-associated molecules.

Further, the following combinations of active agents are also included: a combination of at least one agent that blocks the activity of microglia checkpoint molecules together with at least one agent that enhances the activity of DAM-associated molecules and/or with at least one agent that upregulates the expression of DAM-associated molecules; a combination of at least one agent that reduces the expression of microglia checkpoint molecules together with at least one agent that upregulates the expression of DAM-associated molecules and/or with at least one agent that enhances the activity of DAM-associated molecules.

Finally, any combination of active agents mentioned in the application is considered as part of the invention provided in this application.

The term “treating” as used herein refers to means of obtaining a desired physiological effect. The effect may be therapeutic in terms of partially or completely curing a disease and/or symptoms attributed to the disease. The term refers to inhibiting the disease, i.e. arresting its development; or ameliorating the disease, i.e. causing regression of the disease.

As used herein, the terms “subject” or “individual” or “animal” or “patient” or “mammal,” refers to any subject, particularly a mammalian subject, for whom diagnosis, prognosis, or therapy is desired, for example, a human.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local. In certain embodiments, the pharmaceutical composition is adapted for oral administration.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Preparations for oral administration may be suitably formulated to give controlled release of the active compound.

For buccal administration, the compositions may take the form of tablets or lozenges formulated in conventional manner.

The compositions may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may also be formulated in rectal compositions such as suppositories or retention enemas, e.g., containing conventional suppository bases such as cocoa butter or other glycerides.

For administration by inhalation, the compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

The determination of the doses of the active ingredient to be used for human use is based on commonly used practices in the art, and will be finally determined by physicians in clinical trials. An expected approximate equivalent dose for administration to a human can be calculated based on the in vivo experimental evidence disclosed herein below, using known formulas (e.g. Reagan-Show et al. (2007) Dose translation from animal to human studies revisited. The FASEB Journal 22:659-661). According to this paradigm, the adult human equivalent dose (mg/kg body weight) equals a dose given to a mouse (mg/kg body weight) multiplied with 0.081.

For purposes of clarity, and in no way limiting the scope of the teachings, unless otherwise indicated, all numbers expressing quantities, percentages or proportions, and other numerical values recited herein, should be interpreted as being preceded in all instances by the term “about.” Accordingly, the numerical parameters recited in the present specification are approximations that may vary depending on the desired outcome. For example, each numerical parameter may be construed in light of the number of reported significant digits and by applying ordinary rounding techniques.

The term “about” as used herein means that values of 10% or less above or below the indicated values are also included.

### EXAMPLES

### Materials and Methods

### Animals

Heterozygous 5XFAD transgenic mice (on a C57/BL6-SJL background) that overexpress familial AD mutant forms of human APP (the Swedish mutation, K670N/M671L; the Florida mutation, I716V; and the London mutation, V717I) and PS1 (M146L/L286V) transgenes under the transcriptional control of the neuron-specific mouse Thy-1 promote (5XFAD line Tg6799; The Jackson Laboratory), and male Heterozygous mSOD1 G93A mice on a C57BL/6J background, were taken throughout adulthood in different time points as indicated in the text. Mice were bred and maintained by the animal breeding center of the Weizmann Institute of Science. All experiments detailed herein complied with the regulations formulated by the Institutional Animal Care and Use Committee (IACUC) of the Weizmann Institute of Science.

### Tissue Harvesting

Mice were transcardially perfused with PBS before tissue extraction. Single-cell suspensions of tissues were achieved using software-controlled sealed homogenization system (Dispomix; http://www.biocellisolation.com) in PBS, followed by density gradient separation; Pellet was mixed with 40% percoll and centrifuged in 800G for 20 min at 12° C. Supernatant was discarded and pellet taken further for antibody staining. All Samples were blocked with Fc-block CD16/32 (BD Biosciences, San Jose, Calif.). The following antibodies were used according to the manufacturer's protocol: Brilliant-violet-421 CD45 (1:150, 30-F11, Biolegend Inc.), FITC CD11b (1:100, M1/70, Biolegend Inc.), APC CD11b (1:100, M1/70, eBioscience), PerCP Cy5.5 Gr-1 (1:100, RB6-8C5, eBioscience), Biotin CD11c (1:100, N418, Biolegend Inc.), APC/Cy7 Streptavidin (1:100, Biolegend Inc.). All samples were filtered through a 70-μm nylon mesh before sorting. Cell populations were sorted with SORP-aria (BD Biosciences, San Jose, Calif.).

### Single Cell Sorting

For the sorting of whole immune cell populations, samples were gated for CD45⁺ (Briliant-violet-421, 1:150, 30-F11, biolegend Inc. San Diego, Calif.), after exclusion of doublets. For the isolation of CD11c positive microglia, samples were gated positive for CD45 (Briliant-violet-421, 1:150, 30-F11, Biolegend Inc.), CD11b (APC or FITC, 1:100, M1/70, Biolegend Inc), and CD11c (Biotin CD11c, 1:100, N418, Biolegend Inc, followed by APC/Cy7 Streptavidin, 1:100, Biolegend Inc.), while excluding Gr-1 positive cells (PerCP Cy5.5, 1:100, RB6-8C5, eBioscience) and doublets. Isolated cells were single cell sorted into 384-well cell capture plates containing 2 μl of lysis solution and barcoded poly(T) reverse-transcription (RT) primers for single-cell RNA-seq (28). Four empty wells were kept in each 384-well plate as a no-cell control during data analysis. Immediately after sorting, each plate was spun down to ensure cell immersion into the lysis solution, snap frozen on dry ice, and stored at −80° C. until processed.

To record marker level of each single cell, the FACS Diva 7 “index sorting” function was activated during single cell sorting. Following the sequencing and analysis of the single cells, each surface marker was linked to the genome wide expression profile. This methodology was used to optimize the gating strategy.

### Massively Parallel Single-Cell RNA-seq Library Preparation (Mars-seq)

Single-cell libraries were prepared as previously described (28). In brief, mRNA from cell sorted into cell capture plates are barcoded and converted into cDNA and pooled using an automated pipeline. The pooled sample is then linearly amplified by T7 in vitro transcription, and the resulting RNA is fragmented and converted into a sequencing-ready library by tagging the samples with pool barcodes and illumina sequences during ligation, RT, and PCR. Each pool of cells was tested for library quality and concentration is assessed as described earlier (28).

### Analysis of Single Cell RNA-seq Data

MARS-seq libraries, pooled at equimolar concentrations, were sequenced using an Illumina NextSeq 500 sequencer, at a sequencing depth of 50K-100K reads per cell. Reads are condensed into original molecules by counting same unique molecular identifiers (UMI). We used statistics on empty-well spurious UMI detection to ensure that the batches we used for analysis showed a low level of cross-single-cell contamination (less than 3%).

MARS-seq reads were processed as previously described (27). Mapping of reads was done using HISAT (version 0.1.6); reads with multiple mapping positions were excluded. Reads were associated with genes if they were mapped to an exon, using the UCSC genome browser for reference. Exons of different genes that shared genomic position on the same strand were considered a single gene with a concatenated gene symbol. Cells with less than 1000 UMIs were discarded from the analysis. Genes with mean expression smaller than 0.005 UMIs/cell or with above average expression and low coefficient of variance (<1.2) were also discarded.

### Graph-Based Clustering Analysis

In order to assign cells to homogeneous clusters we used the PhenoGraph clustering algorithm (Levine et al., Data-Driven Phenotypic Dissection of AML Reveals Progenitor-like Cells that Correlate with Prognosis. Cell 162, 184-197 (2015)). Low-level processing of MARS-seq reads results in a matrix U with n rows and m columns, where rows represent genes and columns represent cells. Entry Uij contains the number of unique molecular identifiers (UMIs) from gene i that were found in cell j. PhenoGraph first builds a k-Nearest Neighbors (kNN) graph using the Euclidean distance (k=30) and then refines this graph with the Jaccard similarity coefficient, where the edge weight between each two nodes is the number of neighbors they share divided by the total number of neighbors they have (Levine et al., supra). To partition the graph into modules/communities PhenoGraph uses the Louvain Method (Levine et al., supra). P-values for differential expression analysis between different clusters were calculated using the Mann-Whitney U test (matlab R2016a ranksum function).

### Graph Projection

Graph is visualized in two dimensions using ‘force-directed layout’ (Matlab R2016a graph plot function). Using attractive forces between adjacent nodes and repulsive forces between distant nodes.

### Pseudo-Temporal Ordering of Single Cells (Along Disease Progression Axis)

To obtain pseudo-temporal ordering of the cells along the transition from homeostatic microglia into DAM we used a similar approach to Wanderlust. Cells were represented as nodes in a k-nearest neighbor graph. We selected a representative homeostatic microglia cell (from the homeostatic microglia cluster) and a representative DAM cell as waypoints. For each cell we calculated the shortest path to each of the waypoints and ordered the cells according to their distance. The final trajectory is an average over all graph trajectories. One-dimensional PCA trajectory resulted in a very similar path, suggesting that the majority of the variability inherent in our dataset can be represented by a single principal component.

To estimate the pseudo-time transition point for each gene we sorted the cells according to their temporal ordering. For each gene, we fitted a generalized logistic function (Richards' curve) to the one dimensional vector of UMI counts using nonlinear optimization algorithm (matlab R2016a fmincon function). Genes were ordered according to time to half maximal response (t_{1/2}).

### Immunohistochemistry

Mice were perfused with PBS prior to brain tissue fixation. Microglia from AD and WT mice underwent tissue processing and immunohistochemistry on floating sections (30 μm thick). The following primary antibodies were used: Armenian Hamster anti-CD11c (1:50; Novus, Littleton, Colo.), rabbit anti-IBA1 (1:100, Wako, Richmond, Va.), mouse anti-Aβ 1-16 (1:200, Biolegend, San Diego, Calif.), mouse anti-TIMP2 (1:25, Abcam, Cambridge, Mass.). Secondary antibodies were Cy2/Cy3/Cy5 donkey anti-goat/mouse antibodies (1:150; Jackson ImmunoResearch, West Grove, Pa.) or biotin-SP-conjugated goat anti-Armenian Hamster following cy2/cy3 conjugated streptavidin (1:150; Jackson ImmunoResearch, West Grove, Pa.). The slides were exposed to Hoechst nuclear staining (1:4000; Invitrogen Probes, Carlsbad, Calif.) for 30 sec, prior to their sealing. Staining with secondary antibody alone was used as a negative control. Microscopic analysis was performed using confocal microscopy (Zeiss, LSM880). For quantification of IBA-1+ CD11c+ microglia from cortical slices, IBA-1+ and IBA-1+ CD11c+ cells were counted manually using ImageJ (805 IBA-1+ cells in AD and 139 IBA-1+ in the WT).

Single-Molecule Fluorescent in Situ Hybridization (smFISH)

5XFAD and WT 6-month old female mice were perfused with PBS. Brain tissues harvested and fixed in 4% paraformaldehyde for 3 hours; incubated overnight with 30% sucrose in 4% paraformaldehyde and then embedded in OCT. 6-10 μm cryosections were used for hybridization. Probe libraries were designed and constructed as previously described. Single molecule FISH probe libraries consisted of 48 probes of length 20 bps. Lpl, Cx3cr1, and CSF1 probes were coupled to cy5 or alexa594. Hybridizations were performed overnight in 30° C. DAPI dye for nuclear staining was added during the washes. To detect myeloid plaques, αAβ immunohistochemistry staining was performed simultaneously with the hybridization. Images were taken with a Nikon Ti-E inverted fluorescence microscope equipped with a x60 and x100 oil-immersion objective and a Photometrics Pixis 1024 CCD camera using MetaMorph software (Molecular Devices, Downington, Pa.). Quantification was done manually and calculated according to proximity to the Aβ (AD-Aβ+ and AD-Aβ−). AD-Aβ+ were considered areas that had more than one plaque with a diameter over 35 μm in a field of x60 magnification. smFISH molecules were counted only within the DAPI staining of the cell; a cell was considered Cx3cr1-positive if it had more than 4 Cx3cr1 molecules over 20 0.3 μm Z-stacks. The number of molecules of Lpl, Csf1 was counted over 20 or 21 0.3 μm Z-stacks and normalized to 20 Z-stacks. The p-values were achieved using Mann-Whitney U test (matlab R2016a ranksum function).

Chromatin Immunoprecipitation (iChiP)

iChIP was prepared as previously described (53). Briefly, for chromatin analysis, cells were cross-linked for 8 min in 1% formaldehyde and quenched for 5 min in 0.125 M glycine prior to sorting. Cells were sorted using the described sorting strategy for CD11c positive microglia. Sorted and frozen cell pellets were lysed in 0.5% SDS and sheared with the NGS Bioruptor Sonicator (Diagenode). Sheared chromatin was immobilized on 15 μl Dynabeads Protein G (Invitrogen) with 1.3 μg of anti-H3 antibody (ab1791). Magnetized chromatin was then washed with 10 mM Tris-HCl supplemented with 1X PI. Chromatin was end repaired, dA-tailed and ligated with sequencing adapters containing Illumina P5 and P7 sequences. Indexed chromatin was pooled and incubated with 2.5 μg H3K4me2 antibody (ab32356) at 4° C. for 3 hr and for an additional hour with Protein G magnetic beads (Invitrogen). Magnetized chromatin was washed and reverse cross-linked. DNA was subsequently purified with 1.65X SPRI and amplified by PCR with 0.5 μM of forward and reverse primers containing Illuminia P5-rd1 and P7-rd2 sequences. Library concentration was measured with a Qubit fluorometer and mean molecule size was determined by TapeStation (Agilent). DNA libraries were sequenced on an Illumina NextSeq 500 or HiSeq with an average of over 10 million aligned reads per replicate.

iChiP Analysis

Reads were aligned to the mouse reference genome (mm9, NCBI 37) using Bowtie2 aligner version 2.2.5 with default parameters. The Picard tool MarkDuplicates from the Broad Institute (http://broadinstitute.github.io/picard/) was used to remove PCR duplicates. For scatterplot, the read density (number of reads in 10 million total reads per 1000 bp) was calculated using a sliding window across the entire genome of with 500 bp overlap. The region intensity was given in log-base2 of the normalized density (log2(x+1)).

### Example 1: Identification of a Unique Microglia Type Associated with ALZHEIMER DISEASE

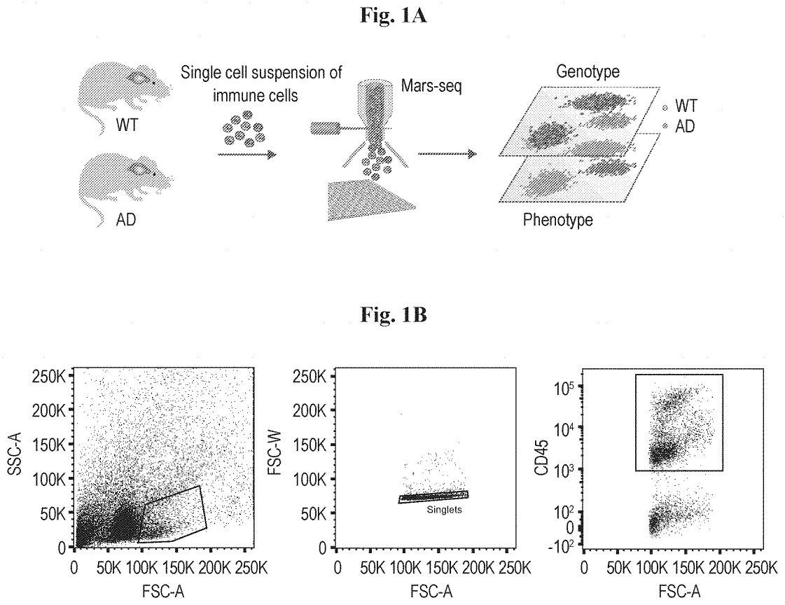

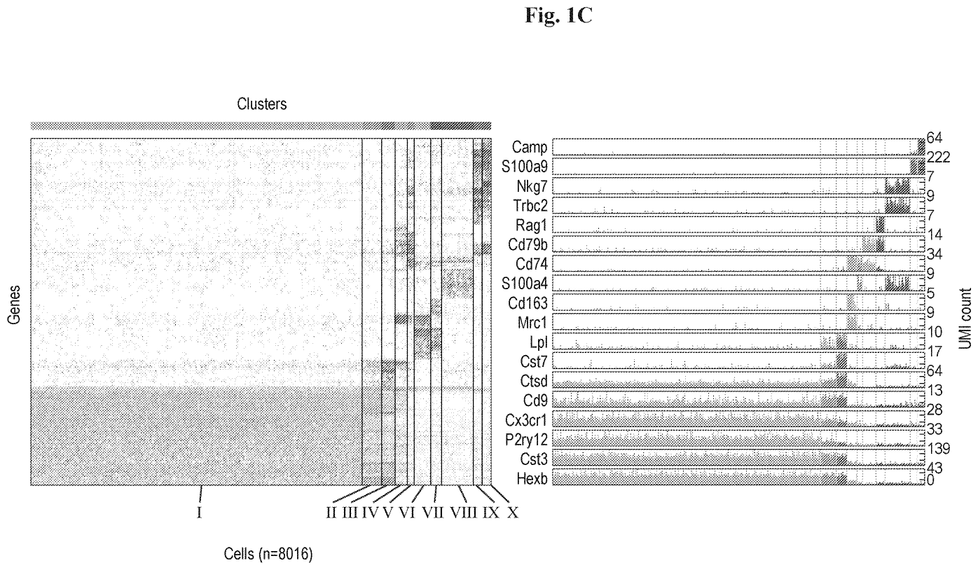

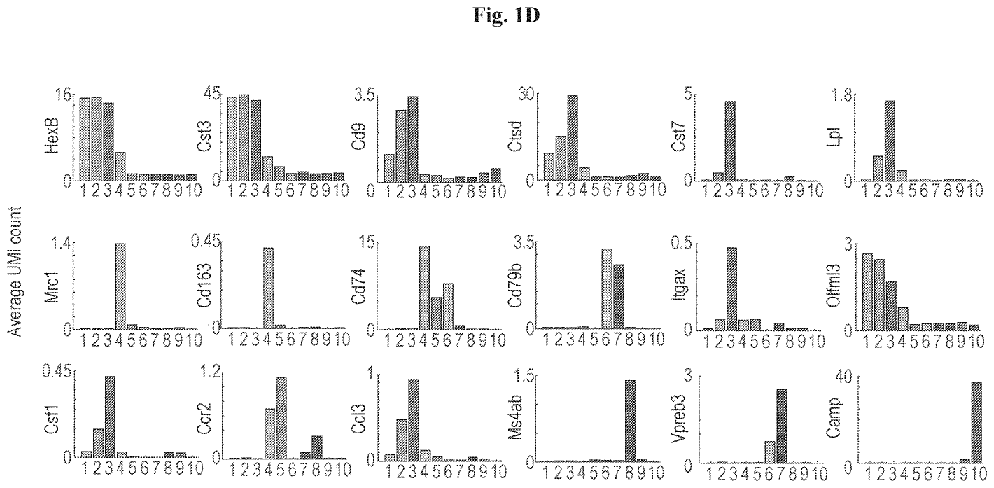

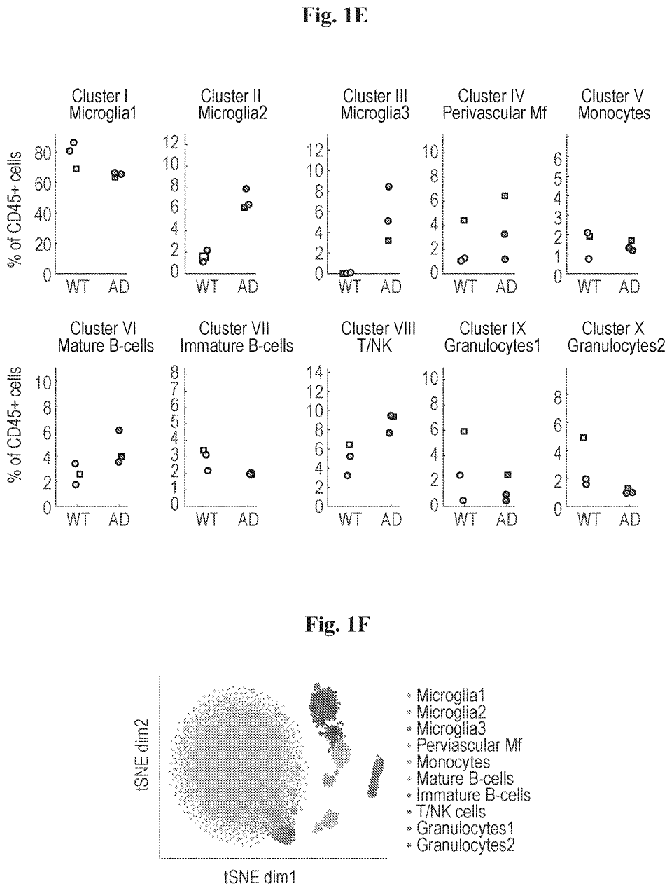

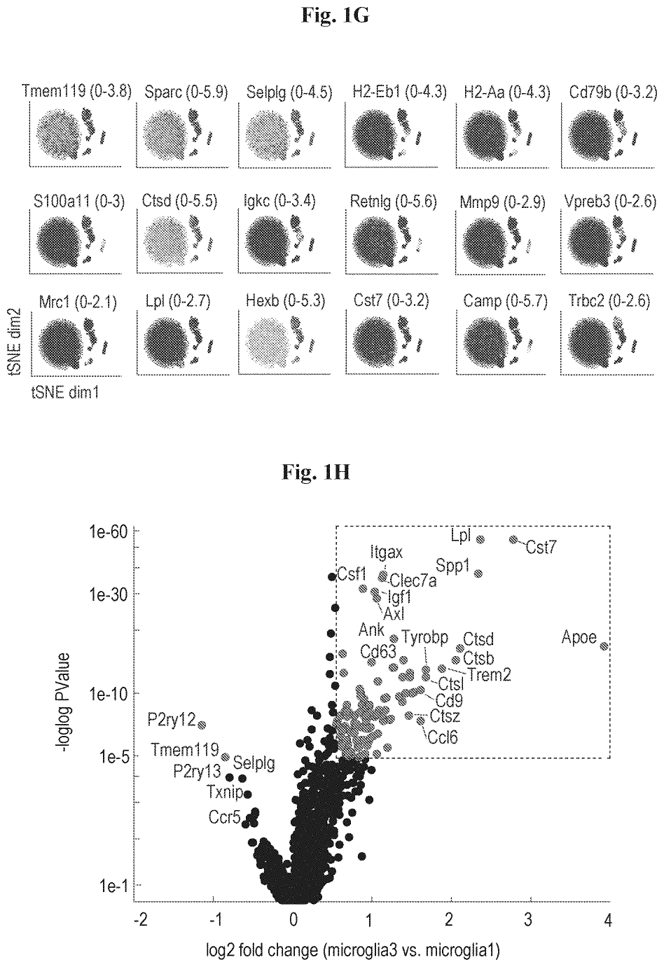

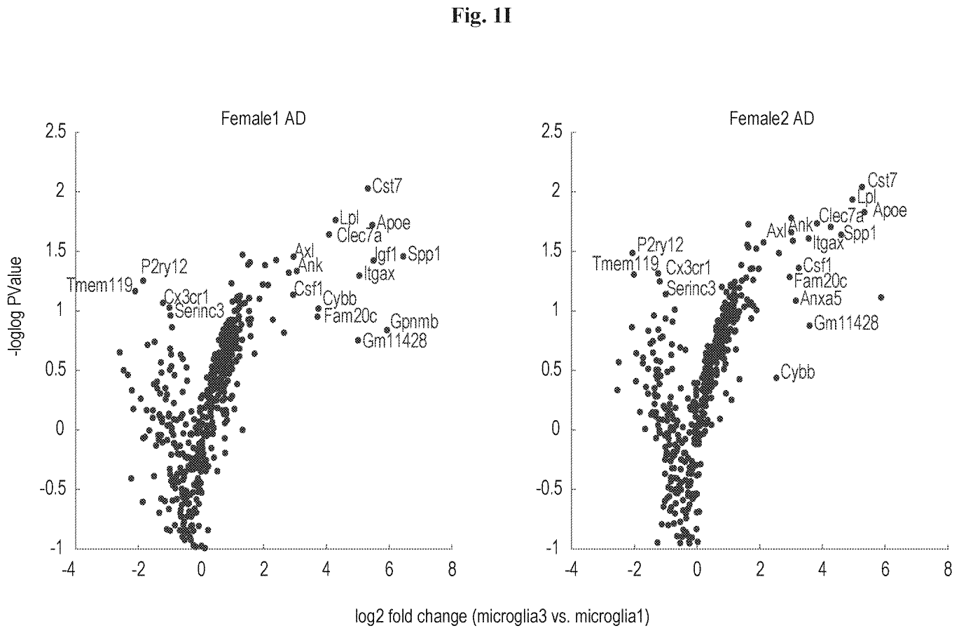

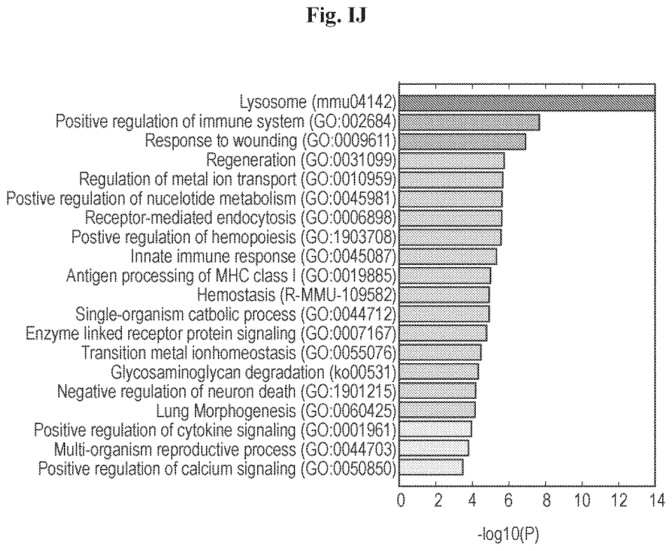

Current characterization of immune cells involved in AD has been obtained from populations sorted according to a small set of canonical cell surface markers. Therefore, the observed gene expression signatures may obscure the presence of additional immune cell types, and overlook the composite picture of related and dynamic subsets in the brain. To de novo characterize the immune cell types and states involved in AD, we first sorted all immune cells (CD45+) from brains of 5XFAD, a commonly used AD transgenic mouse model that expresses five human familial AD gene mutations, compared with age and sex matched wild type controls, and performed massively parallel single-cell RNA-seq (MARS-seq; (28)) (FIGS. 1A, B). In order to link between the canonical surface markers to the genome wide expression profiles, we used an index sorting strategy that allowed for retrospective analysis of surface marker combinations of each individual cell (see Methods). Unsupervised graph-based clustering (PhenoGraph; (Levine et al., see below)) of all 8016 single cells sorted from six wild type and AD mice, created a detailed map of the ten most transcriptionally distinct subpopulations (FIGS. 1C). These distinct immune subpopulations were based on cluster-specific expression patterns of the 500 most variable genes, allowing de novo identification of rare subpopulations. Our analysis identifies a monocyte state, represented in cluster V, a perivascular macrophage group in cluster IV, several lymphocytes sub groups (B cells, T cells, NK cells; clusters VI-VIII), granulocytes (IX-X) and a large group of microglia cells (cluster I), (FIGS. 1C, E). Surprisingly, our analysis also identifies two small groups of cells clusters II (4.2%) and III (2.8%), which that displayed expression of microglial genes (Cst3 and Hexb) with an additional unique signature of lipid metabolism and phagocytic genes such as Apolipoprotein E (Apoe), Lipoprotein lipase (Lpl) and Cystatin F (Cst7, FIGS. 1C, D).

Examining the contribution of wild type versus AD backgrounds to each group of cells revealed a similar percentage of cells in perivascular macrophages, monocytes, group I microglia, granulocytes and a slight differences between the WT and the AD model in lymphocytes (FIG. 1E). Strikingly, group II and III microglia represent distinctive microglia states observed in AD but not in the wild type background, and we define this state as disease associated microglia (DAM). Projection of the cells using t-distributed stochastic neighbor embedding (t-SNE), localizes the DAM group in proximity to the microglia territory and distinct from the monocytes and perivascular macrophages (FIGS. 1F, G). Closer examination of the profiles and key marker genes of homeostatic microglia to group II and III DAM revealed similarity to the microglial program (e.g. Hexb and Cst3, FIGS. 1C, 2C-D). However, DAM also demonstrate significant changes in gene expression compared with microglia such as reduction in the expression levels of several microglia homeostatic genes, including the purinergic receptors P2ry12, P2ry13, Cx3cr1 and Tmem119 (FIGS. 1C, 1H-I), and additional genes: Calm2, Ccr5, Cd164, CD200, Cmtm6, Crybb1, Csf1r, Ecscr, Fscn1, Glul, Gpr56, Ifngr1, Lrba, Lpcat2, Lyn, Maf, Marcks, Olfml3, Pmepa1, Ptgs1, Rhob, Selplg, Serinc3, Slco2b1, Sparc, Srgap2, Txnip, Zfhx3 (Haynes et al., 2006, Mildner et al., 2017). Many more genes are up regulated in DAM including several known AD risk factors such as Apoe, Ctsd, Lpl, Tyrobp (Pottier et al., 2016) and Trem2 (Guerreiro et al., 2013, Jonsson et al., 2013) (FIG. 1H-I). Comparison of homeostatic microglia to group II and III DAM shows that the expression changes for many of the DAM specific genes are in the same trajectory but more pronounced in group III, which may suggest that group II is an intermediate state (also referred to herein as microglia 2) between homeostatic microglia and group III DAM (FIG. 1C and 1F). Gene set enrichment analysis (GO) of DAM-associated genes revealed significant involvement (P<1×10⁻¹⁴) in lysosomal/phagocytic pathways, endocytosis and regulation of the immune response (FIG. 1J).

### Example 2: Disease Associated Microglia Dynamics During AD Progression

AD is a progressive disease with gradual increase in neuronal death and loss of cognitive function. Identifying the relevant changes in DAM regulation along the course of the disease could shed light on the molecular mechanisms of DAM regulation and potentially suggest of new therapeutic targets. AD typically progresses in three general stages; early-stage, mild-moderate, and severe. In the 5XFAD AD model these stages are accelerated and high levels of intraneuronal aggregated (3-amyloid start to appear at around 1.5 months of age and amyloid and plaque deposition at 2-3 months of age. Neuronal loss and deficits in spatial learning initiate at around 6 months and severe cognitive dysfunction is observed at 7-8 months of age. We therefore performed single cell RNA-seq in whole brains of 5XFAD mice at 1, 3, 6 and 8 months of age.

In order to enrich for the rare DAM cells over other immune populations, we used the single cell data to identify potential markers. We identified CD9, Itgax (CD11c), Clec7a and CD63 as potential DAM markers (FIG. 1H), and sorted 1358 CD11c+ microglia from the different time points along disease progression (FIG. 2A). Comparing CD11c+ sorted microglia from 6-month old AD mice with the corresponding CD45+ selection revealed 6.3X enrichment of DAM (FIG. 2B, C). Index sorting of CD11b cells demonstrate that there are no cells with a DAM signature that are CD11c negative (FIG. 2C). However, projection of CD11c+ cells onto our CD45+ map revealed that CD11c+ cells are an heterogonous cell population that include a mixture of various myeloid cells, including microglia, perivascular macrophages and monocytes (FIG. 2D), which may limit biological relevance of bulk analysis using CD11c as a marker for AD. We therefore in silico removed all myeloid contaminants from the time course data and analyzed the remaining 893 DAM and microglial cells. To model the dynamics of microglia along disease progression we generated a k-nearest neighbors graph (kNN) of microglia cells from all major disease stages. Two dimensional projection of the graph identifies homeostatic microglia and DAM on the two extremes of the graph with an intermediate group of cells connecting the two states (FIG. 2E). This analysis demonstrated that microglia in the AD model, and not in the WT, display a transition from homeostatic microglia to DAM population as a function of disease progression (FIGS. 2E, 2F, 2K). While most genes do not change their expression as a function of microglia transition (Hexb), some genes display a decrease in gene expression along this activation axis (Cx3cr1), and some show an increase in their gene expression (Apoe, Lpl, Cd9, Cst7, Trem2) (FIGS. 2G, 2H). In order to define the temporal transcriptional changes leading to DAM formation we generated a mathematical model based on a generalized logistic function to describe the temporal changes in gene expression (see Methods). Our model depicts the molecular order of events in which microglia are switching from the homeostatic state to DAM (FIG. 2I). Analysis of genes in the different disease stages reveals that initial transition events are more frequent in earlier disease stages (3 months) and include down regulation of P2ry12/13 and Cx3cr1 and up regulation of Tyrobp and Apoe. Later regulatory events are more frequent in advanced stages of the disease (8 months), and include up regulation of Cst7, Lpl and Trem2 (FIG. 2I, J). Chromatin plays a major regulatory role in cell-type—specific functions and response.

Transcriptional changes from latent enhancers, rather than previously established enhancers, could resolve whether the DAM program is an immediate transcriptional response or a less anticipated response that requires more complex chromatin rearrangements. We therefore used a high sensitivity method for chromatin immunoprecipitation followed by sequencing (iChIP) comparing microglia and DAM enhancers in wild type and AD mouse model. We observed a highly similar (r²=0.86) global pattern of histone 3 lysine 4 di methyl regions (H3K4me2), which marks promoter and enhancer regions. Focusing on DAM specific genes we observed active H3K4me2 regions in both the microglia and DAM demonstrating that the DAM program is already primed in homeostatic microglia (FIG. 2L).

### Example 3: DAM are Localized Near AD Plaques

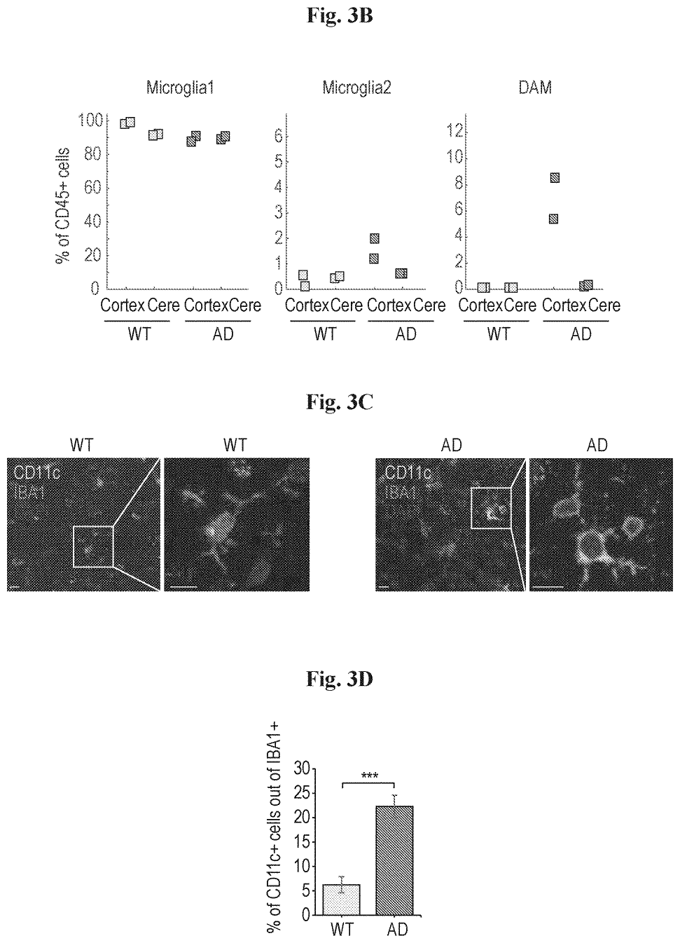

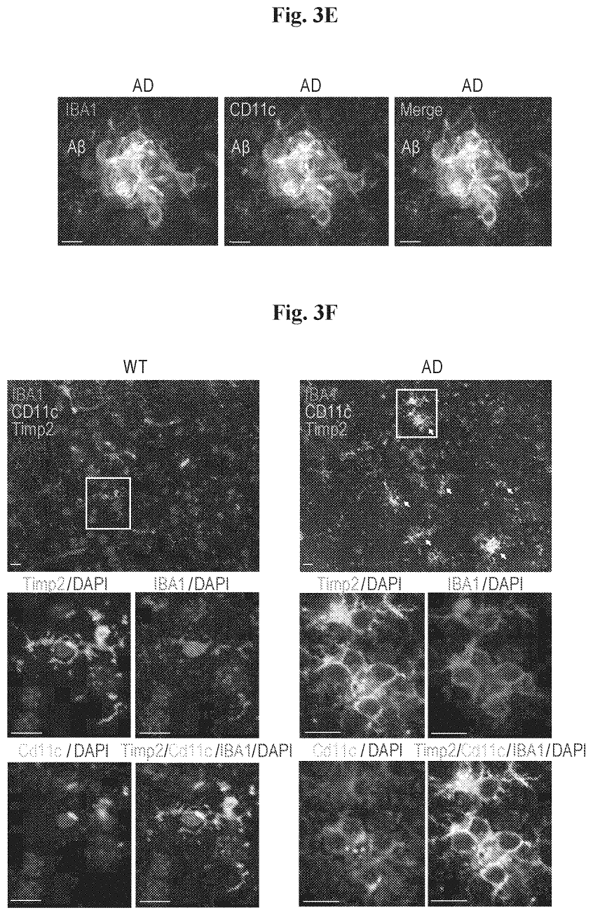

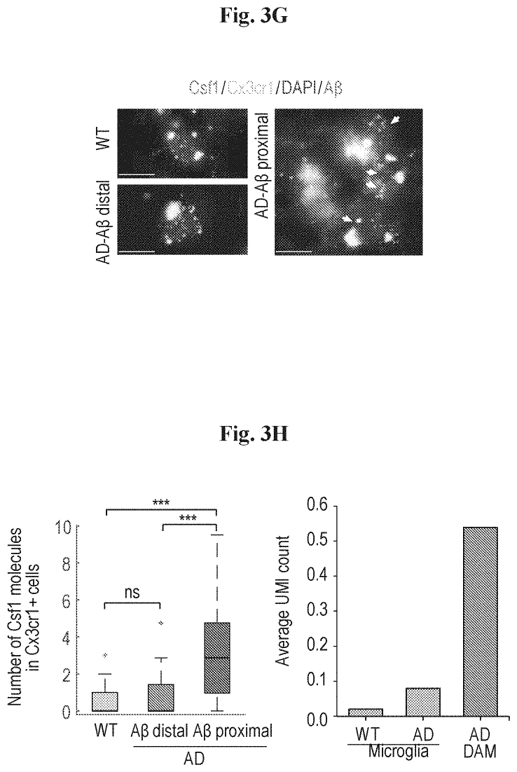

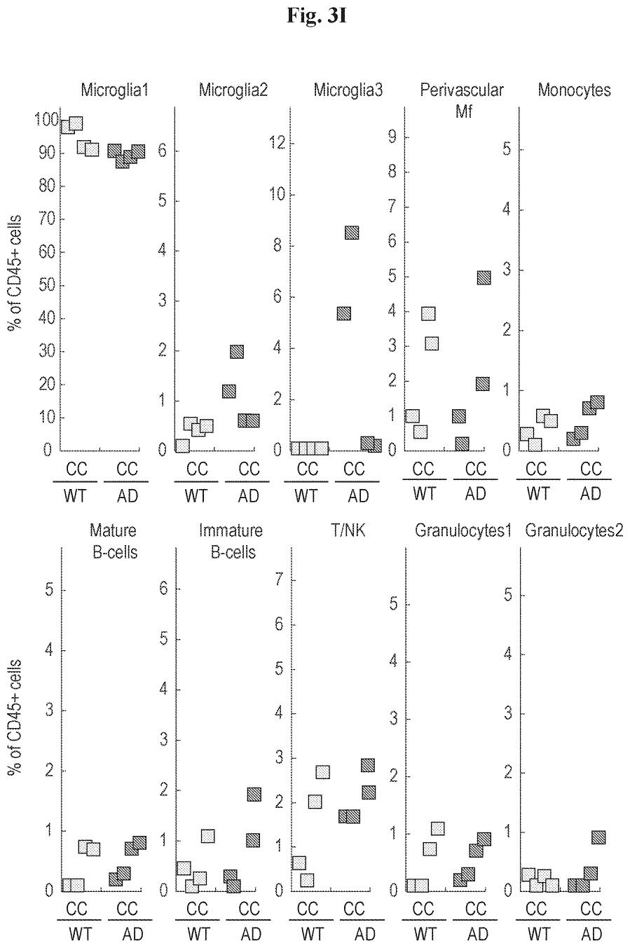

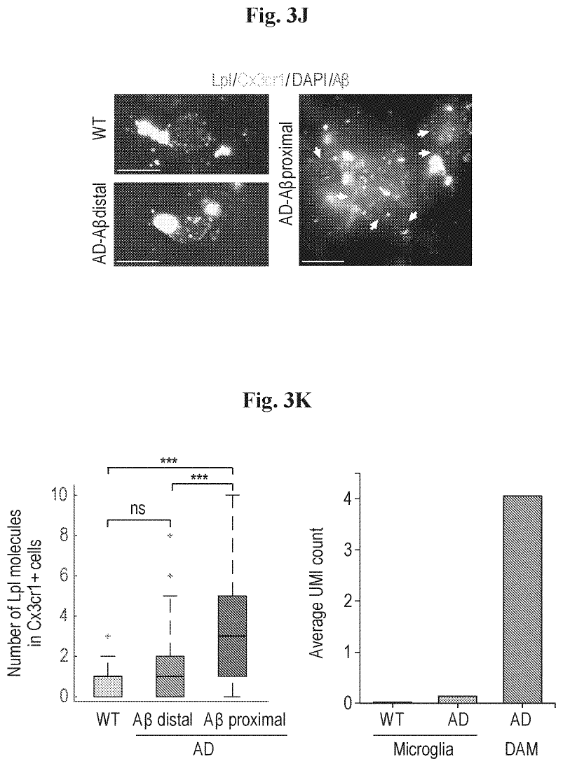

Our single cell analyses were obtained from immune cells isolated from whole brains including the meninges and parenchyma of AD involved (e.g. Cortex) and uninvolved (e.g. Cerebellum) brain regions. In order to spatially orient the immune cell compositions within different brain regions we repeated the single cell sorting experiments on dissected cortex and cerebellum of 6-month old AD and wild type mice and compared the immune composition of these two regions using single cell RNA-seq of all immune (CD45+) cells. Analyzing 6347 cells from four AD and wild type mice, we found that DAM are located within the cortex, but not the cerebellum of AD mice (FIGS. 3A-B, 3I). Using the rich molecular characteristics of DAM we further performed immunohistochemistry staining and single molecule fluorescence in situ hybridization (smFISH). We focused on differentially expressed DAM markers and anti-Aβ plaque labeling. Cortex staining for IBA-1, a classical homeostatic microglial marker, together with CD11c, shows a small overlap (6%) within wild type mice. In contrast, we identified a significant population (22.3%) of microglia that co-expresses these markers in 6-month old AD mice (FIGS. 3C-D). Examining the expression of IBA-1 positive with CD11c positive cells in AD transgenic mice stained for Aβ plaques shows that microglia co-express these markers in proximity to plaque foci (FIG. 3E). Further, CD11c is co-expressed with TIMP2, a marker for DAM, on microglia cells in AD mice (FIG. 3F). Probing for the spatial distribution of DAM specific genes, Csf1 and Lpl, using smFISH, shows significant overlap of these two genes within microglia cells near the Aβ plaques (FIG. 3G, 3H, 3J, 3K). This attributes our identified DAM population to cells localized in the vicinity of the Aβ plaques.

### Example 4: DAM are Phagocytic Cells Conserved in Human and Other Neurodegenerative Diseases

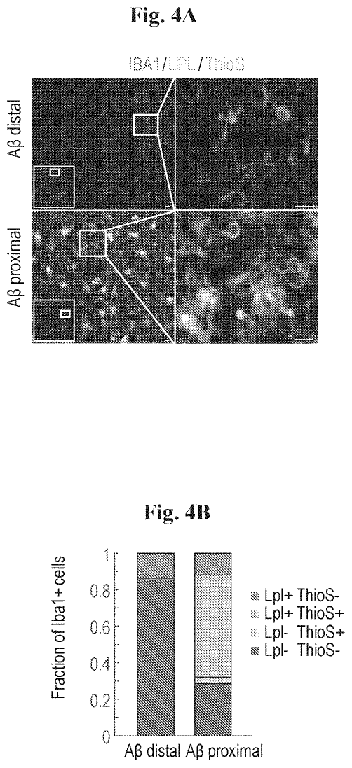

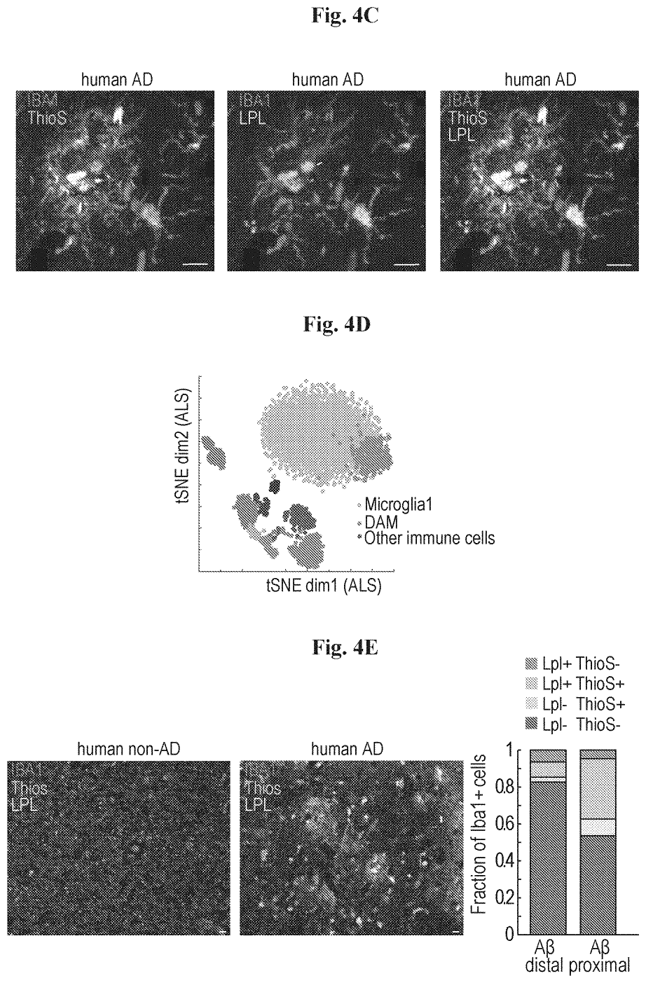

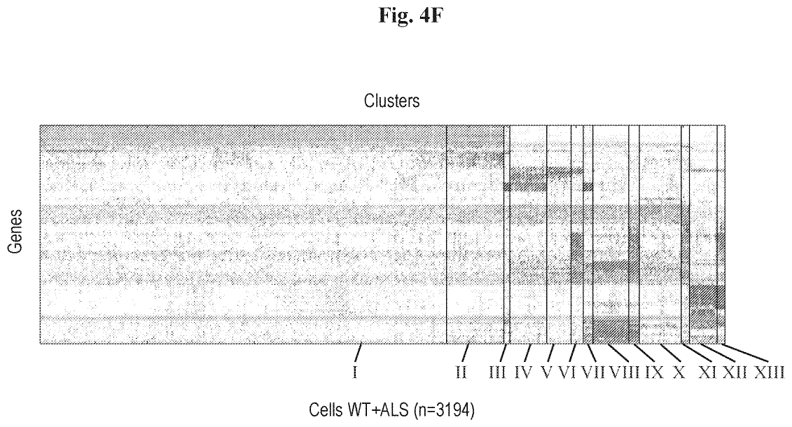

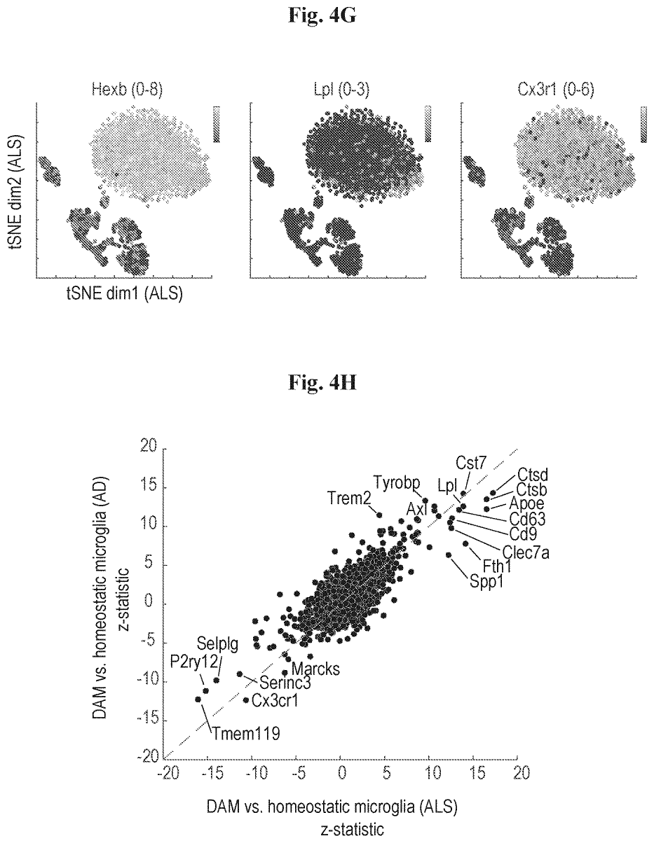

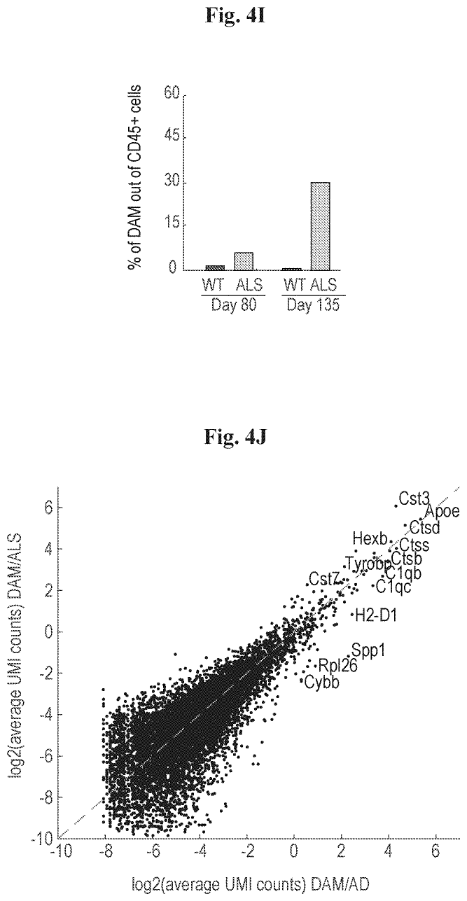

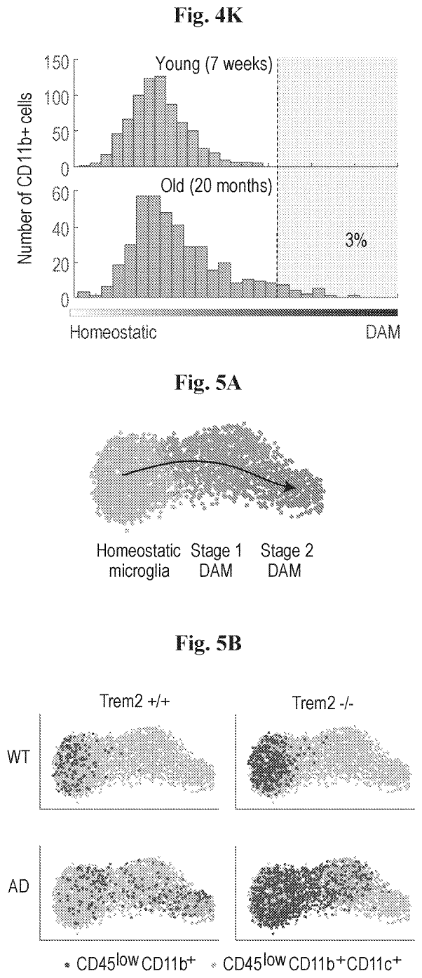

DAM express high levels of phagocytic and lipid metabolism pathways. We therefore examined whether DAM would phenotypically show high incidence of intracellular phagocytic particles of Aβ. We immunostained brain slices with Lpl, a DAM specific gene previously identified as an AD risk factor, together with histological staining using Thioflavin-S, which labels both extracellular and intracellular forms of Aβ. Analysis of these brain slices show that microglia containing Thiofalvin-S labeled particles are mostly clustered in close vicinity of Aβ plaques, from 1.8% of microglia in regions with low density of plaques to 60.6% in regions with high density of plaques (FIGS. 4A-B). These phagocytic microglia showed nearly complete co-expression of the DAM marker Lpl (95.8%; FIG. 4B). Further, in order to examine if DAM cells are conserved in humans, we used the same strategy, and stained human postmortem brains and age-matched non-AD controls, for Aβ and Lpl. Our analysis shows a very strong overlap between Lpl-positive microglia, predominantly around Aβ plaques, in AD post mortem brain samples; such cells could not be detected in non-AD brain slices (FIG. 4C, 4E).

To examine the presence of DAM in other neurodegenerative conditions, we performed single cell analysis of all immune cells from spinal cords of a mouse model of ALS, a neurodegenerative disease characterized by the progressive degeneration of motor neurons in the spinal cord. We sorted 3194 CD45+ cells from the spinal cords of mSOD1 (G93A) mice, a transgenic ALS mouse model mimicking the familial human disease, at early (day 80) and late (day 135) disease progression stages and performed single cell RNA-seq. Analysis revealed similar immune cell populations to those identified in the wild type and AD mice with different proportions (FIG. 4F). The majority of the cells were represented by microglia, expressing the relevant markers such as Hexb and Cx3cr1 (FIG. 4G). Importantly, we also observed a distinct group of cells with microglia characteristics in the spinal cords of ALS mice but not in the wild type mice. These cells display an expression profile that is highly similar to the DAM observed in the AD model, including up regulation of Trem2, Tyrobp, Lpl, and Cst7 as well as down regulation of P2ry12 and Cx3cr1 (FIG. 4H). Of the total CD45+ cells, the percentage of DAM increases from 6% at day 80 to nearly 30% by day 135 (FIG. 4I), which points to further accumulation of DAM during ALS progression. To further generalize the regulation of DAM in various neurodegenerative conditions, we performed single cell RNA-seq on 631 CD11b+ immune cells isolated from whole brains of aged (20-month old) mice compared with young (7-week old) mice. Analysis of the microglia cells revealed a large increase of the DAM population in the aged mice compared with young adults, from non-detected to 3%, respectively (FIG. 4K). These results suggest that DAM actively participate in the dismantling and digestion of the amyloid plaques and are conserved from mice to human neuropathology.

### Example 5: DAM Activation is Initiated by a Trem2 Independent Pathway

In order to better understand the regulatory mechanisms of DAM we further analyzed the single cell data, seeking potential regulators that may trigger DAM activation. Among them, we identified Tyrobp (Tyro protein tyrosine kinase binding protein) and Trem2 (triggering receptor expressed on myeloid cells 2), which form a signaling complex; both components are strongly induced in DAM (FIG. 1H) (Wang et al., 2015, Colonna and Wang, 2016, Painter et al., 2015). Mutations in Trem2 were associated with risk factors in AD (Guerreiro et al., 2013, Jonsson et al., 2013, Colonna and Wang, 2016) and Trem2 deficiency in an AD mouse model accelerated Aβ plaque accumulation and neuronal loss (Jay et al., 2015, Wang et al., 2016, Wang et al., 2015, Yuan et al., 2016). To decipher the role of Trem2 in DAM activation we performed single cell RNA-seq of DAM, using CD11c and CD11b enrichments, from whole brains of Trem2^{+/+} 5XFAD and Trem2^{-/-} 5XFAD mice together with matched wild type and Trem2^{-/-} controls, altogether 3864 cells. Following digital sorting and removal of monocytes contaminants, the remaining microglia cells were clustered using a kNN graph and projected on a two-dimensional space, displaying a spectrum of transcriptional states from homeostatic microglia towards the DAM state (FIG. 5A). Similar to our single cell analysis of AD mice (FIG. 1C) we observed three distinct groups: homeostatic, intermediate state microglia, and DAM (FIG. 5A). Interestingly, the intermediate DAM state, which expressed only a partial set of the DAM program, Tyrobp, Apoe, B2m and Ctsd but not the majority of the lipid metabolism and phagocytic pathway genes (e.g. Lpl), was much more abundant in Trem2 knockout experiment.

Overlaying the mice genotype on our model shows that both wild type and Trem2^{-/-} contain only homeostatic microglia, expressing high levels of microglia marker genes e.g. Cst3, Hexb, Cxc3cr1 (FIGS. 5B-C). Trem2^{+/+} 5XFAD displayed a similar DAM program to that observed (2E, 5C). Strikingly, in the Trem2^{-/-} 5XFAD background, the mice brains were completely depleted of DAM and instead accumulated a large number of cells in the displayed an intermediate state, demonstrating that the DAM program progresses through Trem2 dependent and Trem2 independent pathways (FIGS. 5B, D, E G). A similar intermediate DAM state is observed in the Trem2^{+/+} 5XFAD at a smaller frequency (FIG. 5F), but unlike in the Trem2^{-/-} background most cells proceed and activate the full DAM program (FIGS. 5B, D and E). This analysis suggests that DAM are generated through a two-step mechanism of activation of homeostatic microglia (FIG. 6). An initial activation through an unknown mechanism leads to an intermediate state (Stage 1) in a Trem2 independent mechanism, which critically involves reduction in the expression of homeostatic microglia checkpoint genes (e.g. Cx3cr1 and P2ry12/P2ry13) and up regulation of genes such as B2m, Apoe (associated with AD progression (Castellano et al., 2011)), and the Trem2 adaptor Tyrobp (FIGS. 5 and 6). This intermediate DAM program can be further activated by a secondary activation signal that is Trem2 dependent (Stage 2) and involves up regulation of phagocytic and lipid metabolism genes such as Cst7 and Lpl (FIGS. 5 and 6). Interestingly, no cells with a Trem2 dependent program are observed without the Trem2 independent program, which suggests that DAM activation is a mechanistically coupled temporal event that must first be initiated in a Trem2 independent pathway, followed by activation of the Trem2 dependent program.

### Example 6: Treatment with Antagonists of ‘Homeostasis Molecules’ that Restrain Microglial Activities

The goal of this set of experiments is to test whether early administration of antibodies directed against microglial homeostasis molecules such as P2yR12 or Cx3CR1 or TMEM119, would induce activation or proliferation of the DAM subset of microglia, thereby affecting cognitive performance.

5XFAD mice or Tau mice will be treated at the age of 3 and 6 months (before and after onset of clinical symptoms of cognitive dysfunction, respectively) by administration to the CSF of antibodies or small molecule inhibitors of microglial homeostatic molecules such as P2Ry12 or Cx3CR1 or TMEM119. The outcome will be measured in terms of animal behavior, microglial activity and disease pathology, such as plaque formation, change in microglia phagocytic activity, and gliosis. The cognitive performance will be assessed by Morris water maze (MWM) and by disease pathology It is anticipated that the treatment will cause an increase in phagocytic activity, an increase in gliosis, and/or a delay in or reduction of, loss of cognition.

### Example 7: Treatment with Agonists of Molecules that are Highly Expressed by DAM to Induce Activation of these Cells

It is anticipated that expression of Trem2 is a rate limiting step in microglial activation. We therefore anticipate that earlier or stronger induction of Trem2 may provide a beneficial therapy. Such treatments should be beneficial at all stages of the disease.

5XFAD mice or Tau mice will be treated at the age of 3 and 6 months (before and after onset of clinical symptoms of cognitive dysfunction, respectively) by administration to the CSF of antibodies or small molecule agonists of DAM-associated molecules such as Trem2, ApoE3, Cst7, Lpl, or Tyrobp. The outcome will be measured in terms of animal behavior, microglial activity and disease pathology, such as plaque formation, change in microglia phagocytic activity, and gliosis. The cognitive performance will be assessed by Morris water maze (MWM) and by disease pathology It is anticipated that the treatment will cause an increase in phagocytic activity, an increase in gliosis, and/or a delay in or reduction of, loss of cognition.

### REFERENCES

Baruch et al., Breaking immune tolerance by targeting Foxp3(+) regulatory T cells mitigates Alzheimer's disease pathology. Nature communications 6, 7967 (2015).

Baruch et al., PD-1 immune checkpoint blockade reduces pathology and improves memory in mouse models of Alzheimer's disease. Nature medicine, (2016).

Baum et al., Lipoprotein lipase mutations and Alzheimer's disease. Am J Med Genet 88, 136-139 (1999).

Castellano et al., Human apoE isoforms differentially regulate brain amyloid-beta peptide clearance. Sci Transl Med 3, 89ra57 (2011).

Colonna, Y. Wang, TREM2 variants: new keys to decipher Alzheimer disease pathogenesis. Nat Rev Neurosci 17, 201-207 (2016).

Guerreiro et al., TREM2 variants in Alzheimer's disease. N Engl J Med 368, 117-127 (2013).

Haynes et al., The P2Y12 receptor regulates microglial activation by extracellular nucleotides. Nature neuroscience 9, 1512-1519 (2006).

Jay et al., TREM2 deficiency eliminates TREM2+ inflammatory macrophages and ameliorates pathology in Alzheimer's disease mouse models. The Journal of experimental medicine 212, 287-295 (2015).

Jonsson et al., Variant of TREM2 associated with the risk of Alzheimer's disease. N Engl J Med 368, 107-116 (2013).

Lee et al., CX3CR1 deficiency alters microglial activation and reduces beta-amyloid deposition in two Alzheimer's disease mouse models. The American journal of pathology 177, 2549-2562 (2010).

Ma, T. Jiang, L. Tan, J. T. Yu, TYROBP in Alzheimer's disease. Mol Neurobiol 51, 820-826 (2015).

Mildner, H. Huang, J. Radke, W. Stenzel, J. Priller, P2Y12 receptor is expressed on human microglia under physiological conditions throughout development and is sensitive to neuroinflammatory diseases. Glia 65, 375-387 (2017).

Painter et al., TREM2 in CNS homeostasis and neurodegenerative disease. Molecular neurodegeneration 10, 43 (2015).

Pottier et al., TYROBP genetic variants in early-onset Alzheimer's disease. Neurobiology of aging 48, 222 e229-222 e215 (2016).

Song et al., Alzheimer's disease-associated TREM2 variants exhibit either decreased or increased ligand-dependent activation. Alzheimer's & dementia: the journal of the Alzheimer's Association, (2016).

Wang et al., TREM2 lipid sensing sustains the microglial response in an Alzheimer's disease model. Cell 160, 1061-1071 (2015).

Wang et al., TREM2-mediated early microglial response limits diffusion and toxicity of amyloid plaques. The Journal of experimental medicine 213, 667-675 (2016).

Yuan et al., TREM2 Haplodeficiency in Mice and Humans Impairs the Microglia Barrier Function Leading to Decreased Amyloid Compaction and Severe Axonal Dystrophy. Neuron 90, 724-739 (2016).

1-22. (canceled)

23. A method for treating a neurodegenerative disease, comprising administering to an individual in need thereof an active agent that causes an increase in the number of disease-associated microglia (DAM).

24. The method of claim 23, wherein said active agent causes an increase in the number of DAM by releasing or circumventing a restraint imposed on microglia immune activity by at least one microglia checkpoint molecule.

25. The method of claim 24, wherein said increase in the number of DAM is associated with an increase in microglia phagocytic activity.

26. The method of claim 24, wherein said at least one microglia checkpoint molecule is selected from the group consisting of Cx3cr1, Tmem119, P2ry12, P2ry13, CD200, Ccr5, Calm2, Cd164, Cmtm6, Crybb1, Ecscr, Fscn1, Glul, Gpr56, Ifngr1, Lpcat2, Lrba, Lyn, Maf, Marcks, Olfml3, Pmepa1, Ptgs1, Rhob, Slco2b1, Selplg, Serinc3, Sparc, Srgap2, Txnip, and Zfhx3.

27. The method of claim 26, wherein said at least one microglia checkpoint molecule is selected from the group consisting of Cx3cr1, Tmem119, P2ry12, CD200, Ccr5, Cd164, Zfhx3, Srgap2, Txnip, Ifngr1, P2ry13, Fscn1, Rhob, Cmtm6 and Gpr56.

28. The method of claim 24, wherein said active agent releases a restraint imposed on said microglia by blocking or attenuating the activity of said at least one microglia checkpoint molecule.

29. The method of claim 28, wherein said active agent is a binding-molecule capable of selectively binding and blocking or neutralizing at least one specific microglia checkpoint molecule, said binding molecule selected from the group consisting of a small molecule, an antibody, an affibody, a single-domain an antibody (nanobody), a single chain variable fragment (scFv), an affilin, an affimer, an affitin, an alphabody, an anticalin, an avimer, a DARPin, a Kunitz domain peptide and a monobody.

30. The method of claim 29, wherein said binding-molecule is an antibody selected from the group of antibodies consisting of anti-Cx3cr1; anti-Cx3CL1; anti-Tmem119; anti-P2ry12; anti-CD200; anti-CD200R; anti-Ccr5; anti-Cd164; anti-Zfhx3; anti-Srgap2; anti-Txnip; anti-Ifngr1; anti-P2ry13; anti-Fscn1; anti-Rhob; anti-Cmtm6; and anti-Gpr56 antibody.

31. The method of claim 24, wherein said active agent releases a restraint imposed on said microglia by reducing the expressing of said at least one microglia checkpoint molecule.

32. The method of claim 31, wherein said active agent is a nucleic acid molecule that reduces the gene expression level of a at least one gene encoding a microglia checkpoint molecule selected from the group consisting of Cx3cr1; Cx3CL1; Tmem119; P2ry12; CD200; CD200R; Ccr5; Cd164; Zfhx3; Srgap2; Txnip; Ifngr1; P2ry13; Fscn1; Rhob; Cmtm6; and Gpr56.

33. The method of claim 32, wherein said nucleic acid molecule is an shRNA or artificial siRNA molecule comprising a nucleic acid sequence being complementary to a sequence within a nucleic acid sequence encoding said microglia checkpoint molecule, or a nucleic acid molecule encoding said shRNA or artificial siRNA molecule.

34. The method of claim 33, wherein said siRNA or shRNA molecule comprises a nucleic acid sequence being perfectly complementary to a sequence within the nucleic acid sequence encoding said at least one microglia checkpoint molecule.

35. The method of claim 34, wherein said nucleic acid molecule is comprised within a vector.

36. The method of claim 24, wherein said active agent increases the activity of, or upregulates, at least one DAM-associated molecule.

37. The method of claim 36, wherein said active agent is an agonist increasing activity of, or a nucleic acid molecule upregulating at least one gene encoding, at least one DAM-associated molecule selected from the group consisting of Trem2, ApoE3, Cst7, Lpl, Tyrobp, and CD9, but excluding ApoE4.

38. The method of claim 23, wherein said neurodegenerative disease is selected from the group consisting of Alzheimer's disease, amyotrophic lateral sclerosis, Parkinson's disease, Huntington's disease and age-related macular degeneration.

39. The method of claim 38, wherein said disease is Alzheimer's disease.

40. The method of claim 23, wherein said active agent is administered intracranially; intranasally; or into the cerebrospinal fluid (CSF) via intrathecal injection, lumbar puncture (LP), injection through the Cisterna Magna (CM), intracerebroventricular (ICV) injection.

41. A method for treating Alzheimer's disease, comprising administering to an individual in need thereof an antibody against a microglial checkpoint molecule selected from the group consisting of Cx3cr1, Cx3CL1, Tmem119, P2ry12, CD200, CD200R, Ccr5, and P2ry13, wherein said antibody is administered intracranially, intranasally or into the CSF via intrathecal injection, LP, CM, or ICV injection.

42. A method for treating amyotrophic lateral sclerosis, comprising administering to an individual in need thereof an antibody against a microglial checkpoint molecule selected from the group consisting of Cx3cr1, Cx3CL1, Tmem119, P2ry12, CD200, CD200R, Ccr5, and P2ry13, wherein said antibody is administered intracranially, intranasally or into the CSF via intrathecal injection, LP, CM, or ICV injection.
